(19) United States
(12) Patent Application Publication
(10) Pub. No.: US 2016/0030415 A1
(43) Pub. Date: Feb. 4, 2016

(54) TREATMENT AND PROPHYLAXIS OF KIDNEY DISEASES

(71) Applicant: IPCA Laboratories Limited, Mumbai, Maharashtra (IN)

(72) Inventors: Anil PAREEK, Mumbai, Maharashtra (IN); Nitln CHANDURKAR, Mumbai, Maharashtra (IN)

(21) Appl. No.: 14/772,322

(22) PCT Filed: Mar. 6, 2014
(86) PCT No.: PCT/IN2014/000148

§ 371 Date: Sep. 3, 2015

(30) Foreign Application Priority Data
Mar. 6, 2013 (IN) 684/MUM/2013

(51) Int. Cl. A61K 31/4706; A61K 31/55; A61K 38/05; A61K 31/4178; A61K 31/4245

(52) U.S. Cl. CPC A61K 31/4706; A61K 31/4178; A61K 31/4245; A61K 38/05; A61K 31/55

(57) ABSTRACT

A method for treatment and/or prophylaxis of Kidney Diseases of Non-inflammatory etiology in mammals is described herein. The method comprises administering Hydroxychloroquine, its enantiomer or a pharmaceutically acceptable salt thereof or its combinations with at least one drug selected from the group of angiotensin receptor blockers and angiotensin converting enzyme inhibitors. More particularly, a method is provided for treatment and/or prophylaxis of Diabetic Kidney Disease in mammals, which comprises administering a therapeutically effective amount of Hydroxychloroquine, its enantiomer or a pharmaceutically acceptable salt thereof or its combinations with an angiotensin receptor blockers or an ACE inhibitors. Pharmaceutical compositions are provided for the above treatment. Provided is the use of Hydroxychloroquine, its enantiomer or a pharmaceutically acceptable salt thereof or its combinations with at least one drug selected from the group of angiotensin receptor blockers and ACE inhibitors, wherein in the treatment and/or prophylaxis of Kidney Diseases of Non-inflammatory etiology in mammals.

### FIELD OF INVENTION

The present invention relates to a method for treatment and/or prophylaxis of Kidney Diseases of non-inflammatory etiology in mammals, which comprises administering Hydroxychloroquine, its enantiomer or a pharmaceutically acceptable salt thereof or its combinations with at least one drug selected from the group of Angiotensin receptor blockers and Angiotensin converting enzyme inhibitors. Particularly, the invention relates to a method for treatment and/or prophylaxis of Kidney Diseases caused by various etiologies, including, but not limited to, conditions such as Diabetes Mellitus, Hypertension, Hyperlipidemia etc. in mammals, which comprises administering a therapeutically effective amount of Hydroxychloroquine, its enantiomer or a pharmaceutically acceptable salt thereof or its combination with an angiotensin receptor blocker and/or an ACE inhibitor. The invention further describes pharmaceutical compositions made thereof for the above treatment.

### BACKGROUND OF THE INVENTION

Kidney Disease refers to the deterioration of Kidney Function and has a wide range of etiology, including inflammatory and non-inflammatory diseases. While Acute Kidney Disease refers to the rapid loss of kidney function, Chronic Kidney Disease refers to the progressive loss in renal function over a period of time. Diseases of non-inflammatory etiology including Diabetic Nephropathy (DN), Hyperlipidemia, Vascular Diseases like Hypertension are the leading cause of Chronic Kidney Disease and a prominent cause of cardiovascular mortality. Diabetic Nephropathy is a major micro-vascular complication of diabetes caused by various factors such as sustained hyperglycemia, hypertension, dyslipidemia and glomerular hyperfilteration. It is the single leading cause of end stage renal disease in patients starting renal replacement therapy and affects ˜40% of type 1 and type 2 diabetic patients. It is defined by increased urinary albumin excretion in the absence of other renal diseases.

Gross et al., Diabetes Care, 2005, 28:176-188, describe the different stages of Diabetic Nephropathy: Stage 0 refers to patients at increased risk with elevated Glomerular Filtration Rate which may further deteriorate in case of poor metabolic control. Microalbuminuria, also known as incipient nephropathy (Urinary Albumin Excretion≧20 μg/min and ≦199 μg/min) is Stage 1, during which no Glomerular Filtration Rate (GFR) decline is expected and Macroalbuminuria, also known as overt nephropathy (Urinary Albumin Excretion≧200 μg/min) is Stage 2, characterized by a GFR decline of 1.2 ml/min/month in Type I Diabetes Mellitus and a GFR decline of 0.5 ml/min/month in Type 2 Diabetes Mellitus. The GFR decline becomes greater with more advanced diabetic glomerulopathy and is often accompanied by conditions such as Retinopathy and Nephrotic Syndrome. End Stage Renal Disease, which is the last stage of advanced diabetic glomerulopathy, is characterized by GFR<15 mL/min/1.73 m². People with End Stage Renal Disease or Chronic Kidney Failure need to undergo dialysis or kidney transplantation. Further, people with diabetes who receive transplants or undergo dialysis experience will have higher morbidity and mortality because of coexisting complications of diabetes.

Dinneen et al, Arch Intern Med, 1997, 157:1413-1418 and Stehouwer et al, Diabetes, 2002, 51:1157-1165 conclude that independent of the role as a prognostic factor for macroalbuminuria, the presence of microalbuminuria, reflecting a state of generalized endothelial dysfunction, is a risk factor for cardiovascular disease and mortality. Therefore, management of kidney damage at an early stage is also very critical.

Gross et al., Diabetes Care, 2005, 28:176-188, describe that apart from genetic susceptibility, the main, potentially modifiable, risk factors for diabetic nephropathy are sustained hyperglycemia, hypertension, dyslipidemia and glomerular hyperfilteration. Mari-Anne et al, BMJ, 1997; 314:783-8, elucidate that factors such as increased urinary albumin excretion rate, long term poor glycemic control and serum cholesterol concentration predict the development of incipient and overt diabetic nephropathy. Sandor et al, Nephrol Dial Transplant, 2003, 18 [Suppl5]:v21-v23 describe that increased activity of renin-angiotensin-aldosterone system is an important pathogenic factor in the development of nephropathy in diabetic patients. Gross et al., Diabetes Care, 2005, 28:176-188, elucidate the recommended treatment of Diabetic Nephropathy, involving multifactor approach, targeting the main risk factors and renin-angiotensin system blockade using drugs such as Angiotensin Converting Enzyme Inhibitors (ACE Inhibitors) and Angiotensin Receptor II Blockers (ARBs).

However, as pointed out by Jacobsen et al, Curr Opin Nephrol Hypertens., 2004, 13(3):319-24, the current treatment strategies reduce, but do not prevent, the progression of kidney damage. Further, Pantelis et al, American Journal of Hypertension (2008); 21, 8, 922-929 point out that neither ACE Inhibitors nor Angiotensin Receptor Blockers have been able to decrease mortality rate in patients with diabetic nephropathy.

ACE inhibitors are considered the mainstay of therapy in Diabetic Nephropathy in spite of not being able to reduce the mortality. Further, ACE Inhibitors are unable to block Angiotensin II production completely. Francesco et al, Nephron Clin Pract, 2009; 113:c286-293 add that because of the incomplete inhibition of ACE by ACE Inhibitors, the initial decline in angiotensin II and aldosterone levels often gradually returns to pre-treatment levels.

Also, Barry et al, N Engl J Med, 2001; 345:861-9 state that Angiotensin Converting Enzyme Inhibitor treatment has not been able to offer therapeutic benefit in end-stage renal disease in patients with type 2 diabetes, which entails the need for an alternate therapeutic agent in such patients.

The National Collaborating Center for Chronic Conditions, Royal College of Physicians, recommend in Guidelines issued on Type 2 diabetes management, the replacement of ACE Inhibitors with Angiotensin Receptor Blockers if ACE inhibitors are poorly tolerated.

Randomized clinical trials [Jacobsen et al, Curr Opin Nephrol Hypertens., 2004, 13(3):319-24, Mogensen et al, BMJ, 2000; 321(7274): 1440-1444 and Hughes et al, Pharmacotherapy, 2005, 25(11):1602-20.] have reported that dual blockade of renin-angiotensin system using angiotensin converting enzyme inhibitors and angiotensin II receptor blockers may prove to be more effective in treatment of albuminuria and elevated blood pressure level than monotherapy with either class of drugs alone.

However, the higher efficacy in case of dual blockade using angiotensin converting enzyme inhibitors and angiotensin II receptor blockers remains questionable as highlighted by the IMPROVE Study, wherein after 20 weeks of follow-up, the changes in the geometric mean of albumin excretion rate from baseline were similar in groups administered with either Ramipril plus Irbesartan or Ramipril alone.

Several side effects have also been observed on co-administration of ACEI and ARB. The ONTARGET trial reported an increased incidence of clinically important renal dysfunction (death, doubling of serum creatinine, or dialysis) in telmisartan and ramipril combination treatment groups compared with groups receiving telmisartan alone or ramipril alone. Also, those receiving combination therapy suffered from hypotensive symptoms more frequently than those receiving monotherapy. This acute hypotension may increase the risk of myocardial ischemia in patients with narrowed coronary vessels.

The use of such combinations at their therapeutic levels may also be associated with a decline in the hematocrit in patients suffering from renal impairment. Therefore, the use of this therapy approach in patients already suffering from impaired renal function may expose them to serious risks of hematological reactions. Further, as elucidated by Francesco et al, Nephron Clin Pract 2009, 113:c286-293, though the combination therapy is claimed to have additional renoprotective effects, it can have deleterious effects on renal function, especially in patients with mild proteinuria.

Diuretics, Beta Blockers and Calcium Channel Blockers are among the other treatments prescribed in case ACE Inhibitors and Angiotensin Receptor Blockers cannot be tolerated. Treatment with thiazide diuretics mainly aims at reducing hypertension and has been associated with hypokalemia, hyponatraemia, volume depletion, hypercalcaemia, and hyperuricaemia. As elucidated in the UMHS 2008 guidelines, the efficacy of beta blockers and calcium channel blockers in preventing kidney damage is low and some members of the dihydropyridine class of calcium channel blockers (e.g., nifedipine, felodipine) may, in fact, increase urinary albumin excretion, and should be avoided in patients with microalbuminuria. As Smith et al, Kidney Int. 1998; 54:889-96 point out, the dihydropyridine class of calcium channel inhibitors is not effective in treatment of nephropathy and the non-dihydropyridine class of calcium channel inhibitors fails to reduce the rate of decrease of glomerular filtration rate with its use.

The compound Hydroxychloroquine and its pharmaceutical salts are described in U.S. Pat. No. 2,546,658. EP588430 describes the use of S enantiomer of the compound in treatment of rheumatoid arthritis and Systemic Lupus Erythematosus.

US2011230501 describes the use of pharmaceutical compositions comprising Hydroxychloroquine in reduction of hyperglycemia and management of diabetes mellitus.

Willis et al, Lupus, 2012, 21(8):830-5 describe the results of LUMINA, a, multi-ethnic US cohort study, elucidating the effect of Hydroxychloroquine treatment on pro-inflammatory cytokines and disease activity in Systemic Lupus Erythematosus. Wasko et al, JAMA, 2007, 298(2):187-93 state that the use of Hydroxychloroquine reduces the risk of diabetes in patients suffering from rheumatoid arthritis. Walalce et al, Am J Med, 1990, 89(3):322-6 and Munro et al, Ann Rheum Disease, 1997, 56(6):374-7 have highlighted the beneficial effect of Hydroxychloroquine on serum lipids.

Fessler et al, Arthritis & Rheumatism, 2005; 52:1472-1480 report a correlation between use of anti-malarials and occurrence of Systemic Lupus Erythematosus (SLE). Further, Siso et al, Lupus, 2008, 17, 281-288 disclose that previous exposure to antimalarials in patients suffering from lupus nephritis was negatively associated with development of renal failure and resulted in a better survival rate compared to those never treated with anti-malarials. Pons-Estel et al, Arthritis & Rheumatism, 2009; 61: 830-839, disclose the possible protective effect of Hydroxychloroquine in retarding renal damage in patients suffering from Lupus Nephritis) which is a complication of Systemic Lupus Erythematosus, an auto-immune disorder leading to chronic inflammation. The treatment for Lupus Nephritis mainly comprises corticosteroid therapy and administration of immunosuppressive agents.

Thus, it can be seen that there is a need for effective treatment for the recovery of renal function in diabetic nephropathy or other kidney disease conditions of non-inflammatory etiology. Available treatment options mainly arrest further progression of renal damage, however, at the cost of adverse reactions. Therefore, there is a need in the art to find a better medication for the treatment of Diabetic Nephropathy and other kidney diseases of non-inflammatory etiology, not only to offer better recovery of renal function but also to provide a safe and effective treatment. This forms the object of the present invention.

### SUMMARY OF THE INVENTION

Unexpectedly, it has now been found by applicants that Hydroxychloroquine, its enantiomer or the Drug Combinations comprising Hydroxychloroquine or its enantiomer can be used for recovery of renal function, especially in Kidney Diseases of non-inflammatory etiology including, but not limited to, Kidney Diseases caused by Type 1 and 2 Diabetes Mellitus, Systemic infections, Drug-Induced Nephropathies, Generalized Tubulopathies such as Fanconi syndrome, Acute Tubular Necrosis or tubular atrophy, vascular diseases such hypertension, hypertensive nephropathy or nephrosclerosis, interstitial fibrosis, glomerular alterations and periglomerular fibrosis caused by benign arterial hypertension, Dyslipidemia, hyperlipidemia, hyperlipidemic nephropathy, glomerulosclerosis or Nephrosis caused by persistent filtration of lipids and/or lipoproteins, Renal Artery Disease, Microangiopathy and Idiopathic Kidney Diseases.

Hydroxychloroquine (Figure I), chemical name being, 2-[{4-[(7-chloroquinolin-4-yl)amino]pentyl}(ethyl)amino]ethanol, is a hydroxy derivative of Chloroquine.

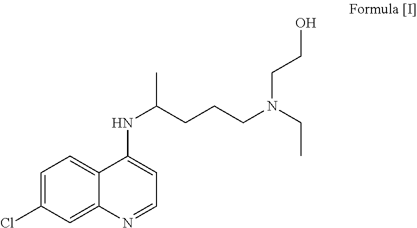 [SMILES: CCN(CCO)CCCC(C)Nc1ccnc2cc(Cl)ccc12]

The surprising results obtained according to the present invention have important clinical implications for treatment or prevention of progression of renal diseases, especially renal diseases caused by Diabetes or others as described before. Thus, kidney diseases of non-inflammatory etiology, especially, Diabetic Nephropathy may now be effectively treated with Hydroxychloroquine, its enantiomer or a pharmaceutically acceptable salt thereof, or its combinations with at least one drug selected from the group comprising Angiotensin receptor blockers (ARB) and Angiotensin converting enzyme (ACE) inhibitors, with lower side effects than those attributable to the current therapy for the disease.

Accordingly, the present invention provides a method for treatment and/or prophylaxis of Kidney Diseases of non-inflammatory etiology including, but not limited to, Kidney Diseases caused by Type 1 and 2 Diabetes Mellitus, Systemic infections, Drug-Induced Nephropathies, Generalized Tubulopathies such as Fanconi syndrome, Acute Tubular Necrosis or tubular atrophy, Vascular Diseases such as hypertensive nephropathy or nephrosclerosis, interstitial fibrosis, glomerular alterations and periglomerular fibrosis caused by benign arterial hypertension, Dyslipidemia, hyperlipidemia, hyperlipidemic nephropathy, glomerulosclerosis or Nephrosis caused by persistent filtration of lipids and/or lipoproteins, Renal Artery Disease, Microangiopathy and Idiopathic Kidney Diseases. The said method comprises administering Hydroxychloroquine, its enantiomer or a pharmaceutically acceptable salt thereof or its combinations with at least one drug selected from the group comprising Angiotensin receptor blockers and Angiotensin converting enzyme inhibitors. Further, the kidney diseases according to the present invention are characterized by conditions including but not limited to, abnormal albumin: creatinine ratio, albuminuria including microalbuminuria and macroalbuminuria, abnormalities in glomerular filtration rate, higher serum creatinine levels, higher total protein in kidney homogenate, increased blood urea nitrogen levels, hypoproteinemia, abnormalities in the urinary sediment, abnormal results on kidney imaging studies and/or end stage renal failure.

In one aspect, the present invention provides a method for treatment and or prophylaxis of Diabetic Kidney Disease in a mammal comprising administration of a therapeutically effective amount of Hydroxychloroquine, its enantiomer or a pharmaceutically acceptable salt thereof. In a preferred embodiment, the present invention provides a method of treatment and/or prophylaxis of different stages of Diabetic Kidney Disease including but not limited to Microalbuminuria, Macroalbuminuria, and End Stage Renal Disease. In another embodiment, the present invention provides a method for primary and secondary prophylaxis of Diabetic Kidney Diseases in a mammal by administering a therapeutically effective amount of Hydroxychloroquine, its enantiomer or a pharmaceutically acceptable salt thereof.

In one embodiment, the present invention provides a method for treatment and/or prophylaxis of Kidney Diseases caused by Hypertension and/or Hyperlipidemia in a mammal by administering a therapeutically effective amount of Hydroxychloroquine, its enantiomer or a pharmaceutically acceptable salt thereof.

In another embodiment, the present invention provides a method for the treatment or prevention of, and to delay the progression of kidney diseases of non-inflammatory etiology in a mammal, which comprises administering to the mammal a Drug Combination containing a therapeutically effective amount of Hydroxychloroquine, its enantiomer or a pharmaceutically acceptable salt thereof and at least one drug selected from the group comprising Angiotensin receptor blockers (ARB) and Angiotensin converting enzyme (ACE) inhibitors. The individual drug may be administered simultaneously, separately or sequentially for a suitable period in such a manner to get an additive and synergistic effect.

In another aspect, the present invention provides pharmaceutical composition(s) or kit comprising hydroxychloroquine, its enantiomer or its pharmaceutically acceptable salts and at least one ARB or ACE inhibitors for simultaneous, separate or sequential use, especially in the treatment and/or prevention and to delay progression of kidney diseases, in particular, but not limited to, different stages of Diabetic Nephropathy such as Microalbuminuria, Macroalbuminuria and End Stage Renal Disease. Hydroxychloroquine or its enantiomer combination with ARB or ACE inhibitor may be formulated in a single dosage form or formulated individually and packed in a kit or may be provided as separate compositions.

In a preferred embodiment, Angiotensin Receptor Blockers may be selected from, but not limited to, Losartan, Azilsartan, Irbesartan, Candesartan, Olmesartan, Valsartan and the like, preferably the ARB drug is Losartan or Azilsartan. ACE inhibitors may be selected from, but not limited to, Ramipril, Lisinopril, Perindopril, Captopril, Enalapril, Quinapril, Benazepril, Imidapril, Zofenopril, Trandolapril and the like, preferably the ACE inhibitor drug is Ramipril, Lisinopril or Benazepril.

The details of one or more embodiments of the invention are set forth in the description below. Other features, objects and advantages of the invention will be apparent from the appended examples and claims.

### BRIEF DESCRIPTION OF THE DRAWINGS

FIG. 1-8 show the effect of Hydroxychloroquine and its combination with Losartanon various physiological parameters indicative of recovery from renal damage in Diabetic rats.

FIG. 1 shows the effect of Hydroxychloroquine and its combination with Losartan on Thiobarbituric Acid Reactive Substance level in Kidney Homogenate as seen in study 1.

FIG. 2 shows the effect of Hydroxychloroquine and its combination with Losartan on Glutathione (GSH) level in Kidney Homogenate as seen in study 1.

FIG. 3 shows the effect of Hydroxychloroquine and its combination with Losartan on Total Protein in Kidney Homogenate as seen in study 1.

FIG. 4 shows the effect of Hydroxychloroquine and its combination with Losartan on Blood Glucose levels as seen in study 1.

FIG. 5 shows the effect of Hydroxychloroquine and its combination with Losartan on Serum Creatinine levels as seen in study 2.

FIG. 6 shows the effect of Hydroxychloroquine and its combination with Losartan on Total serum protein levels as seen in study 2.

FIG. 7 shows the effect of Hydroxychloroquine and its combination with Losartan on TBARS levels in Kidney Homogenate as seen in study 2.

FIG. 8 shows the effect of Hydroxychloroquine and its combination with Losartan on Total Protein in Kidney Homogenate as seen in study 2.

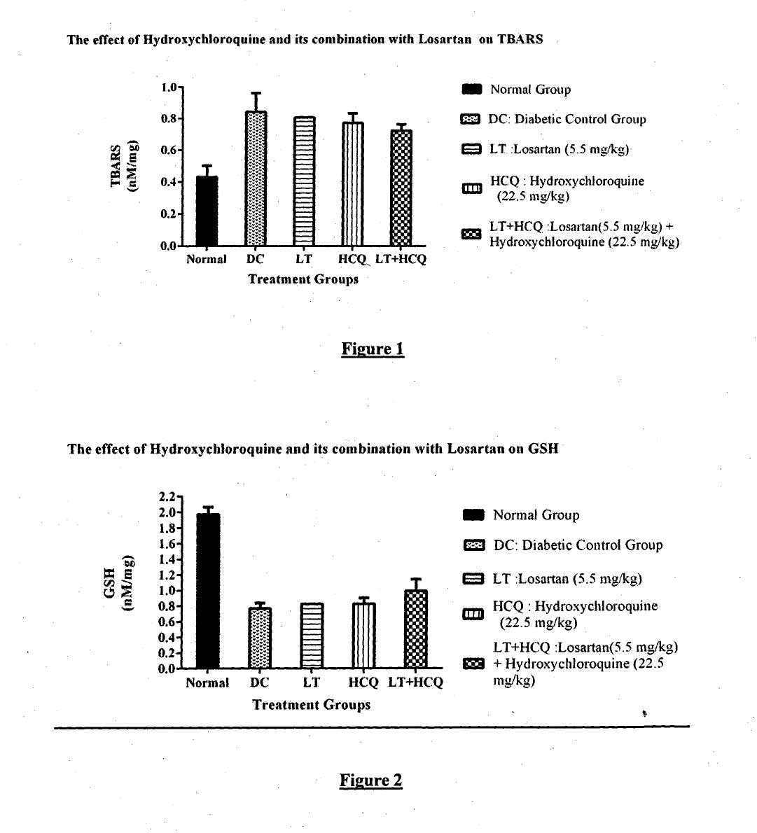

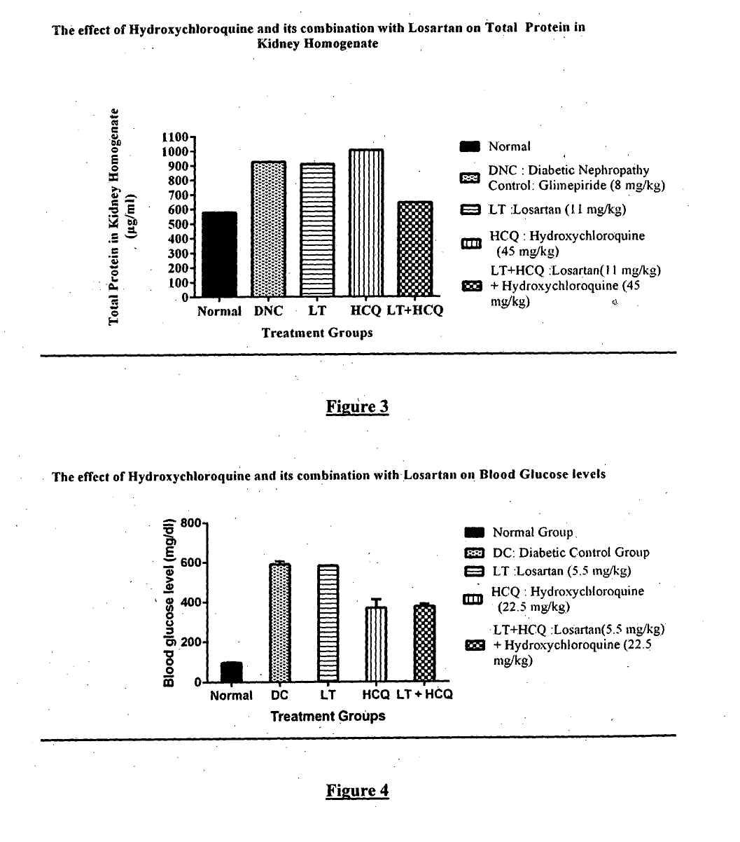

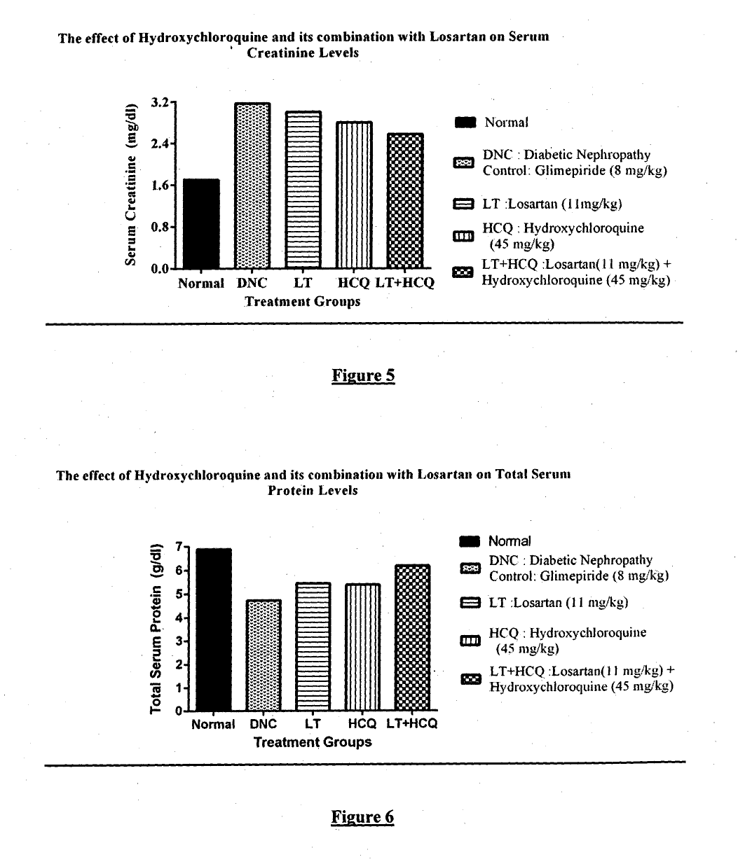

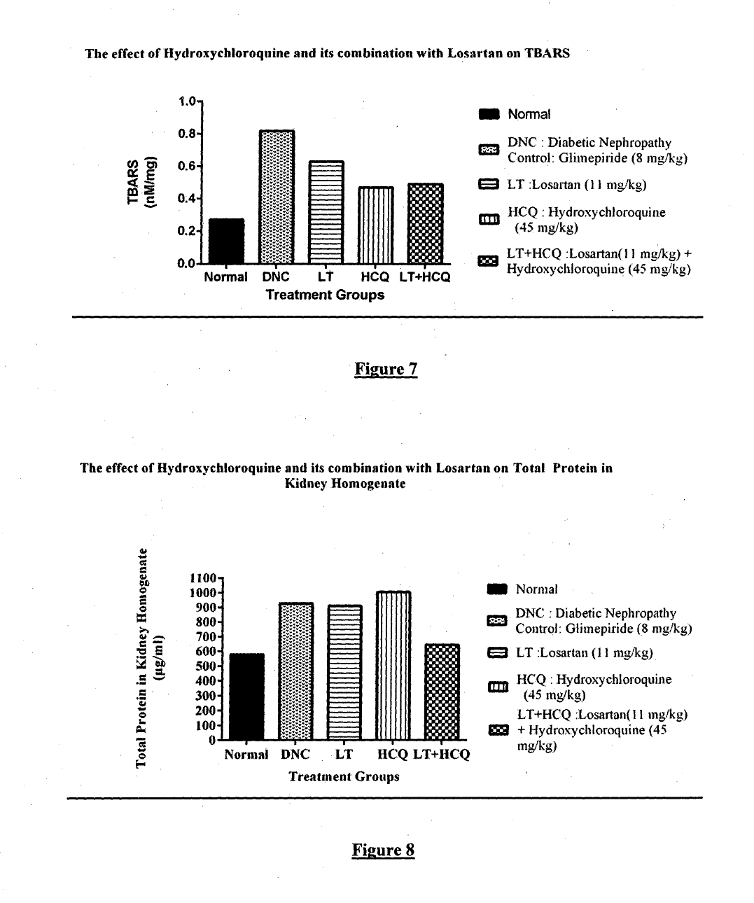

### DETAILED DESCRIPTION OF THE INVENTION

Unless specified otherwise, all technical and scientific terms used herein have the same meaning as commonly understood by one of ordinary skill of the art, to which this invention belongs. Although any methods and materials similar or equivalent to those described herein can be used in the practice or testing of the present invention, the preferred methods and materials are described. To describe the invention, certain terms are defined herein specifically as follows:

Unless stated to the contrary, any of the words “comprising”, “includes”, “comprising”, and “comprises” mean “comprising without limitation” and shall not be construed to limit any general statement that it follows to the specific or similar items or matters immediately following it. Embodiments of the invention are not mutually exclusive, but may be implemented in various combinations. The described embodiments of the invention and the disclosed examples are given for the purpose of illustration rather than limitation of the invention as set forth in the appended claims.

The term ‘Hydroxychloroquine’ comprises its (R) or (S) enantiomer individually or any mixture of (R) and (S) enantiomers, including a racemic mixture.

The term “kidney diseases of non-inflammatory etiology” refers to kidney diseases due to different etiology except those caused by inflammation.

The term ‘Diabetic nephropathy’ refers to any deleterious effect on kidney structure and/or function caused by diabetes mellitus. More specifically, diabetic nephropathy is differentiated into different stages, the first being that characterized by microalbuminuria (30-300 mg urinary albumin per 24 hours). This may progress to macroalbuminuria, or overt nephropathy (>300 mg urinary albumin per 24 hours). Later still, progressive renal functional decline characterized by decreased Glomerular Filtration Rate (GFR) results in clinical renal insufficiency and End Stage Renal Disease (ESRD). End Stage Renal Disease is characterized by GFR<15 mL/min/1.73 m².

The term ‘patient’ and ‘subject’, whenever referred to herein means mammals including humans requiring medical attention.

The term ‘prophylaxis’ refers to any medical procedure whose purpose is to prevent a disease.

The term ‘primary prophylaxis’ refers to prevention of the development of a disease.

The term ‘secondary prophylaxis’ indicates that the disease has already developed and the patient is protected against worsening of this process).

The term ‘Diabetes mellitus’ refers to a metabolic disorder of multiple etiology characterized by chronic hyperglycaemia with disturbances of carbohydrate, fat and protein metabolism resulting from defects in insulin secretion, insulin action, or both. “Diabetes mellitus” encompasses both the Type 1 (Insulin Dependant Diabetes Mellitus or IDDM) and Type 2 (Non Insulin Dependent Diabetes Mellitus or NIDDM) forms of, the disease. The effects of diabetes mellitus include long-term damage, dysfunction and failure of various organs. Diabetes mellitus may present with characteristic symptoms such as thirst, polyuria, blurring of vision, and weight loss. In its most severe forms, ketoacidosis or a non-ketotic hyperosmolar state may develop and lead to stupor, coma and, in absence of effective treatment, death. Often symptoms are not severe, or may be absent, and consequently hyperglycaemia sufficient to cause pathological and functional changes may be present for a long time before the diagnosis is made. The long-term effects of diabetes mellitus include progressive development of the specific complications of retinopathy with potential blindness, nephropathy that may lead to renal failure, and/or neuropathy with risk of foot ulcers, amputation, Charcot joints, and features of autonomic dysfunction. People with diabetes are at increased risk of cardiovascular, peripheral vascular and cerebrovascular disease.

The term “Diabetic Kidney Disease” refers to kidney disease in subjects suffering from Diabetes Mellitus.

The term “therapeutic effect” is art-recognized and refers to a local or systemic effect in animals, particularly mammals, and more particularly humans caused by a pharmacologically active substance.

The phrase “therapeutically effective amount” means that amount of such a substance that produces some desired local or systemic effect at a reasonable benefit/risk ratio applicable to any treatment. The therapeutically effective amount of each substance will vary depending upon the subject, severity of disease or condition being treated, body weight and age of the subject, the manner of administration and the like, which can readily be determined by one of ordinary skill in the art. For example, certain compositions described herein may be administered in a sufficient amount to produce a desired effect at a reasonable benefit/risk ratio applicable to such treatment.

It has now surprisingly been found that it is possible to treat Kidney Diseases of non-inflammatory etiology in mammals using Hydroxychloroquine or its enantiomer, or a pharmaceutically acceptable salt thereof which may provide a better treatment option or ameliorate the drawbacks of the current treatment. The present invention thus provides a safe and effective method for treatment of Kidney Diseases of non-inflammatory etiology including, but not limited to, Kidney Diseases caused by Type 1 and 2 Diabetes Mellitus, Systemic infections, Drug-Induced Nephropathies, Generalized Tubulopathies such as Fanconi syndrome, Acute Tubular Necrosis, or tubular atrophy, Vascular Diseases such as hypertensive nephropathy or nephrosclerosis, interstitial fibrosis, glomerular alterations and periglomerular fibrosis caused by benign arterial hypertension, Dyslipidemia, hyperlipidemia, hyperlipidemic nephropathy, glomerulosclerosis or Nephrosis caused by persistent filtration of lipids and/or lipoproteins, Renal Artery Disease, Microangiopathy and Idiopathic Kidney Diseases. The various aspects of the invention are described in detail with specific embodiments/conditions hereafter.

In accordance with one aspect, the present invention provides a method for the treatment and/or prophylaxis of Kidney Diseases of non-inflammatory etiology characterized by conditions including, but not limited to, abnormal albumin:creatinine ratio, albuminuria including microalbuminuria and macroalbuminuria, abnormalities in glomerular filtration rate, higher serum creatinine levels, higher total protein in kidney homogenate, increased blood urea nitrogen levels, hypoproteinemia, abnormalities in the urinary sediment, abnormal results on kidney imaging studies and/or end stage renal failure. The said method comprises administering therapeutically effective amount of Hydroxychloroquine, its enantiomer or a pharmaceutically acceptable salt thereof or its combinations with at least one drug selected from the group comprising Angiotensin receptor blockers (ARB) and Angiotensin converting enzyme (ACE) inhibitors. In one embodiment, the present invention provides a method for the treatment and/or prophylaxis of Kidney Diseases of non-inflammatory etiology including, but not limited to, Kidney Diseases caused by Type 1 and 2 Diabetes Mellitus, Systemic infections, Drug-Induced Nephropathies, Generalized Tubulopathies such as Fanconi syndrome, Acute Tubular Necrosis, Vascular Diseases such as Hypertension, Renal Artery Disease, Microangiopathy and Idiopathic Kidney Diseases by administering therapeutically effective amount of Hydroxychloroquine, its enantiomer or a pharmaceutically acceptable salt thereof or its combinations with at least one drug selected from the group comprising Angiotensin receptor blockers (ARB) and Angiotensin converting enzyme (ACE) inhibitors.

In accordance with one aspect, the present invention provides a method for the treatment and/or prophylaxis of different stages of Diabetic Kidney Diseases, including but not limited to Microalbuminuria, Macroalbuminuria and End Stage Renal Disease, as well as compositions for use in such methods. The method according to the present invention comprises administering therapeutically effective amounts of Hydroxychloroquine, its enantiomer or a pharmaceutically acceptable salt thereof or its combinations with at least one drug selected from the group comprising Angiotensin receptor blockers (ARB) and Angiotensin converting enzyme (ACE) inhibitors. Treatment according to the present invention shows marked reduction in serum creatinine levels and renal oxidative stress, thus indicative of recovery of renal function. Hydroxychloroquine or its enantiomer's therapy also lowers the blood glucose levels and increase the total protein levels in the serum.

In one aspect, the present invention provides a method for the treatment and/or prophylaxis of Kidney Diseases of non-inflammatory etiology by administering therapeutically effective amount of Hydroxychloroquine, its enantiomer or a pharmaceutically acceptable salt thereof or its combinations with at least one drug selected from the group comprising Angiotensin receptor blockers (ARB) and Angiotensin converting enzyme (ACE) inhibitors, characterized in that the Kidney Disease is of non-inflammatory etiology.

In another aspect, the present invention provides a method for treatment of diabetic nephropathy in a mammal comprising administering therapeutically effective amount of Hydroxychloroquine, its enantiomer or a pharmaceutically acceptable salt thereof or its combinations with at least one drug selected from the group comprising Angiotensin receptor blockers (ARB) and Angiotensin converting enzyme (ACE) inhibitors. In one embodiment, the present invention provides a method for treatment of different stages of diabetic nephropathy including, but not limited to, Microalbuminuria, Macroalbuminuria and End Stage Renal Disease. In a preferred embodiment, the invention provides a method for primary prophylaxis of kidney diseases of non-inflammatory etiology in subjects at increased risk, characterized by increase in Glomerular Filtration Rate. In, another embodiment, the present invention provides a method for treatment and/or prophylaxis of Microalbuminuria caused by Diabetes Mellitus in a mammal, characterized by Urinary Albumin Excretion≧20 μg/min and ≦199 μg/min. In a preferred embodiment, the present invention provides a method for treatment and/or prophylaxis of Macroalbuminuria induced by Diabetes Mellitus in a mammal characterized by Urinary Albumin Excretion≧200 μg/min and by a GFR decline of 1.2 ml/min/month in Type I Diabetes Mellitus and a GFR decline of 0.5 ml/min/month in Type 2 Diabetes Mellitus. In a preferred embodiment, the present invention provides a method of Prophylaxis of End Stage Renal Disease induced by Diabetes Mellitus in mammals characterized by GFR<15 mL/min/1.73 m², wherein such patients require renal replacement therapy. In clinical practice, the Glomerular Filtration Rate (GFR) is considered a good indicator for estimation of overall kidney function. Glomerular Filteration Rate, in turn, is estimated based on serum creatinine concentration. The reduction in serum creatinine concentration, therefore, serves as an excellent indicator of recovery of kidney function according to the invention.

In all aspects of the present invention, the methods discussed herein may comprise the step of administration of one or more therapeutic agents. In the cases of drug combinations according to the present invention, the method comprises simultaneous, separate or sequential administration of effective amounts of the above active compounds and/or co-treatment with other medications, in a ratio which provides an additive and synergistic effect.

The specific ARB useful for the drug combinations according to the present invention include, but are not limited to, Losartan, Valsartan, Azilsartan, Candesartan, Telmesartan and the like: Preferably, the ARB is selected from Losartan and Azilsartan. The specific ACE inhibitors useful for the drug combinations according to the present invention include, but are not limited to, Ramipril, Lisinopril, Perindopril, Captopril, Enalapril, Quinapril, Benazepril, Imidapril, Zofenopril, Trandolapril and the like. Preferably, the ACE inhibitor is Ramipril, Lisinopril or Benazepril.

The quantity of each substance to be administered will vary depending upon the subject to be treated, severity, of disease or condition being treated, body weight and age of the subject, the manner of administration and the like, which can readily be determined by one of ordinary skill in the art. The method for oral administration may comprise administration of Hydroxychloroquine, its enantiomer or its pharmaceutically acceptable salt for an average adult, in a quantity range from 50 mg to 800 mg calculated based on the weight of Hydroxychloroquine free base, more preferably a dose of 150 to 400 mg may be administered as a daily dose. The dose of Hydroxychloroquine, its enantiomer or a pharmaceutically acceptable salt thereof may be adjusted lower, if necessary when used in combination with ARB or ACE inhibitor according the present invention. The dose of ARB and ACE inhibitor may be selected based on the response of each drug in the combination for a targeted baseline of parameters indicative of recovery/damage. This may be determined by a physician based on the patient's response to individual drug. In practice, one may start with the lower doses and titrate to get a response based on individual response to the drug combination. Typically, the dose of Valsartan may range from 40 mg to 320 mg, the dose of Candesartan may range from 5 to 40 mg, the dose of Telmisartan may range from 20 to 80 mg, the dose of Losartan may range from 10 mg to 100 mg, the dose of Azilsartan may range from 10 to 100 mg, the dose of Ramipril may range from 1 mg to 100 mg, the dose of Lisinopril may range from 5 to 80 mg, the dose of Captopril may range from 10-150 mg, the dose of Perindopril may range from 1-10 mg, the dose of Enalapril may range from 1 to 40 mg, the dose of Quinapril may range from 1 to 80 mg, the dose of Benazepril may range from 1 to 80 mg.

The doses discussed herein are for a patient of 60 kg average body weight. It should be understood that dose may be adjusted with respect to body weight of the patient, health/condition of the patient, severity of the disease and metabolic profile of the compounds of the present invention. The skilled person in the art has the ability and expertise to adjust the dosage as required.

According to another aspect of the present invention, there are provided combinations/compositions for use in the methods described herein. For the avoidance of any doubt, where reference is made to the administration of an amount of active ingredient, this may be comprised within the composition of this aspect of the invention. In accordance with the above aspect, the invention provides a pharmaceutical combination/compositions comprising Hydroxychloroquine, its enantiomer or a pharmaceutically acceptable salt thereof and at least one drug selected from the group consisting of Angiotensin receptor blockers and Angiotensin converting enzyme inhibitors, for the management or treatment or prophylaxis of kidney disease, wherein, the kidney disease is of non-inflammatory etiology.

In a preferred aspect of the present invention, the invention provides pharmaceutical combination comprising Hydroxychloroquine, its enantiomer and its combination with other drugs comprising Angiotensin Receptor Blockers, such as Losartan which provide a markedly higher decrease in serum creatinine levels, renal oxidative stress parameters as well as blood glucose levels. Such drug combinations are therefore, act synergistically as the ideal drug candidates for the treatment of Diabetic Nephropathy. In a preferred embodiment, there is provided a fixed dose composition of Hydroxychloroquine or its enantiomer with Losartan, characterized in that said composition comprises a dose of 20-100 mg of Losartan or a pharmaceutically acceptable salt thereof. In another preferred embodiment, there is provided a fixed dose composition of Hydroxychloroquine or its enantiomer with Azilsartan, characterized in that said composition comprises a dose of 10-100 mg of Azilsartan or a pharmaceutically acceptable salt thereof. In yet another embodiment, there is provided a fixed dose composition of Hydroxychloroquine or its enantiomer with Ramipril, characterized in that said composition comprises a dose of 1-100 mg of Ramipril or a pharmaceutically acceptable salt thereof. In a further embodiment, there is provided a fixed dose composition of Hydroxychloroquine or its enantiomer with Lisinopril, characterized in that said composition comprises a dose of 1-100 mg of Lisinopril or a pharmaceutically acceptable salt thereof. In yet another embodiment there is provided a fixed dose composition of Hydroxychloroquine or its enantiomer with Benazepril, characterized in that said composition comprises a dose of 1-100 mg of Benazepril or a pharmaceutically acceptable salt thereof.

The fixed dose pharmaceutical compositions of the invention are preferably administered orally on a daily basis as an immediate release or modified release dosage form. The dosage form of the present invention may be formulated as a single dosage unit, as two separate unit dosages, and/or in any form of the many variations known in the art, which include, but are not limited to, tablets, pills, hard capsules, soft capsules, pharmaceutical sachets and powders for reconstitution.

The formulations of the invention may further contain water insoluble permeable polymers, herein defined as “modified release polymers”, to adjust their release profile. These polymers may either be coated onto formulations such as tablets, microgranules, capsules or pills, or be mixed together with the other ingredients of any of the formulations listed above.

In one embodiment, the pharmaceutical compositions of the present invention are provided in the form of tablets prepared by mixing the active agents with excipients. Typical excipients include diluents, fillers, binders, lubricants, disintegrants, glidants, colorants, pigments, taste making agents, modified release polymers, sweetners, plasticizers, and any acceptable auxiliary substances such as absorption enhancers, penetration enhancers, surfactants, co-surfactants, and specialized oils. Examples of excipients include calcium phosphates, such as dibasic calcium phosphates, anhydrous dibasic calcium phosphate, tribasic calcium phosphate, etc.; microcrystalline cellulose, powdered cellulose; starch, pre-gelatinized starch; sodium starch glycolate; dexterates; mannitol, sorbitol; povidone; ethyl cellulose; lactose; kaolin; silicic acid; lubricants such as magnesium stearate, calcium stearate, stearic acid, mineral oil, glycerin, sodium lauryl sulfate, polyethylene glycol; and/or talc. Sodium starch glycolate, talc and the lubricant magnesium stearate may be used to prepare compositions of the present invention to aid in tablet manufacture. A premix of Hydroxychloroquine or its enantiomer and one or more active agents including Angiotensin receptor blockers and Angiotensin converting enzyme inhibitors may be obtained by mixing the said compounds with ingredients and thereafter either directly compressing the mixture into tablets or filling said mixture into capsules optionally along with other suitable ingredients to obtain final dosage form. A unit dosage comprising the free form of Hydroxychloroquine or its enantiomer may be obtained as a granular premix by suitably processing that compound with acceptable ingredients such as polymers, which can be directly compressed or formulated with additional excipients.

The compositions and methods of the present invention may be employed in the treatment and/or prophylaxis of Kidney Diseases of non-inflammatory etiology including, but not limited to, Kidney Diseases caused by Type 1 and 2 Diabetes Mellitus, Systemic infections, Drug-Induced Nephropathies, Generalized Tubulopathies such as Fanconi syndrome, Acute Tubular Necrosis, Vascular Diseases such as Hypertension, Renal Artery Disease, Microangiopathy and Idiopathic Kidney Diseases. The compositions and methods of the present invention may further be employed for treatment and/or prophylaxis of different stages of Kidney Diseases caused by Diabetes Mellitus in mammals, including but not limited to Microalbuminuria, Macroalbuminuria, Proteinuria and End Stage Renal Failure. The compositions and methods of the present invention may further be employed in the prophylaxis and/or treatment of Kidney Diseases of non-inflammatory etiology characterized by conditions including but not limited to abnormal albumin:creatinine ratio, albuminuria including microalbuminuria and macroalbuminuria, abnormalities in glomerular filtration rate, higher serum creatinine levels, higher total protein in kidney homogenate, increased blood urea nitrogen levels, hypoproteinemia, abnormalities in the urinary sediment, abnormal results on kidney imaging studies, and/or end stage renal failure.

In yet another aspect, the invention provides Hydroxychloroquine or its enantiomer or a pharmaceutically acceptable salt thereof for use in the treatment and/or prophylaxis of kidney disease in a human in need thereof, wherein the kidney disease is of non-inflammatory etiology.

In yet another aspect, the invention provides Hydroxychloroquine or its enantiomer or a pharmaceutically acceptable salt thereof for use in the treatment of and/or prophylaxis of kidney disease in a human in need thereof, wherein Hydroxychloroquine or its enantiomer is administered at a dosage of 50-800 mg/day.

In yet another aspect, the invention provides Hydroxychloroquine or its enantiomer or a pharmaceutically acceptable salt thereof and at least one drug selected from the group consisting of Angiotensin receptor blockers and Angiotensin converting enzyme inhibitors, for use in the management or treatment or prophylaxis of kidney disease, wherein, the kidney disease is of non-inflammatory etiology.

For the purpose of the present invention, the Angiotensin Receptor Blocker is selected from the group consisting of Valsartan, Telmisartan, Losartan, Irbesartan, Azilsartan, Olmesartan and the Angiotensin Converting Enzyme Inhibitor is selected from the group consisting of Ramipril, Lisinopril, Perindopril, Captopril, Enalapril, Quinapril, Benazepril, Imidapril, Zofenopril, Trandolapril.

The efficacy and additive as well as synergistic effect of the combination of hydroxychloroquine with an Angiotensin Receptor Blocker is established by the following experiment.

### Pharmacological Evaluation: Experimental Section

The efficacy of drugs according to the invention for treatment and/or prophylaxis of kidney diseases is demonstrated in the following preclinical evaluation attributed to improvements in biochemical parameters such as serum creatinine, blood glucose level and renal oxidative stress parameters including Thiobarbituric acid reactive substances (TBARS), Glutathione (GSH), and Total Protein in Kidney Homogenate.

The methods described in Terri et al, Diabetes Volume, 1997, 46:1612-1618, and Takashi et al, Jpn J. Pharmacol, 1997, 75, 59-64, were followed for inducing Diabetes in rats by injecting Streptozotocin. The Efficacy of the drugs was tested in two different models, one involving comparison with diabetic control (i.e. Diabetic Rats not treated with any medication) and the other involving comparison with the anti-diabetic control group (i.e. Diabetic rats administered Glimepiride) in two separate studies.

Study 1: Experimental Studies to Evaluate Efficacy of Hydroxychloroquine and its Combination with Losartan in Renal Damage

25 male Wistar rats weighing 150-250 g were divided randomly into 5 groups consisting of 5 animals each. Diabetes was induced in rats by single administration of streptozotocin (55 mg/kg/i.p) dissolved in 0.1 M-citrate buffer, pH 4.5. Group 1 (normal control) consisted of normal rats that neither received Streptozotocin (STZ) nor any test drug; Group 2 served as streptozotocin induced diabetic control. Group 3 was diabetic rats treated with Losartan (LT) (5.5 mg/kg); Group 4 was diabetic rats treated with Hydroxychloroquine (HCQ) (22.5 mg/kg), Group 5 was diabetic rats treated with combination of HCQ (22.5 mg/kg) and LT (5.5 mg/kg). The study drugs were dissolved/suspended in distilled water and administered orally once a day for eight weeks. Blood glucose levels and renal oxidative stress parameters including TBARS, GSH and total Protein in Kidney Homogenate were assessed after the completion of eighth week of the study.

The levels of Thiobarbituric Acid Reactive Substance (TBARS) and Total Protein in Kidney Homogenate were significantly increased in diabetic rats as compared to normal controls at the completion of eighth week (p<0.01) (Table 1). In comparison with the diabetic control group, the test drugs caused a significant reduction in the Total Protein in Kidney Homogenate in the following order of efficacy: HCQ-LT combination>>HCQ>LT (p<0.01) (Table 1) such that a synergistic effect was evident in case of HCQ-LT combination. The test drugs also caused a significant reduction in the TBARS levels in comparison with the diabetic control group in the following order of efficacy: HCQ-LT combination>HCQ>LT (p<0.01) (Table 1), with the HCQ-LT combination demonstrating a synergistic effect.

The Glutathione levels were significantly reduced (p<0.01) in diabetic rats as compared to normal controls at the completion of eighth week (Table 1). In comparison with the diabetic control group, the test drugs caused a significant increase in the Glutathione levels in the following order of efficacy: HCQ-LT combination>>HCQ>LT (p<0.01) (Table 1), with the HCQ-LT combination demonstrating a synergistic effect.

Blood Glucose levels were significantly increased (p<0.01) in diabetic rats as compared to normal control at the completion of eighth week (Table 1). In comparison with the diabetic control group, the test drugs caused a significant decrease in blood glucose levels in the following order of efficacy: HCQ>HCQ-LT combination>LT (p<0.01) (Table 1).

TABLE 1
Effect of HCQ and its combination with Losartan on renal oxidative stress
parameters and blood glucose in Streptozocin (STZ) induced diabetic rats

| | | | | Total Protein | |
|---|---|---|---|---|---|
| | | | | in Kidney | |
| | | TBARS | GSH | Homogenate | Blood glucose |
| | Treatment | (nM/mg) | (nM/mg) | (μg/ml) | level (mg/dl) |
| | Dose | Mean ± SEM | Mean ± SEM | Mean ± SEM | Mean ± SEM |
| Group | (mg/kg) | (% Naive) | (% Naive) | (% Naive) | (% Control) |
| Normal | — | 0.433 ± 0.07 | 1.973 ± 0.09 | 575.75 ± 11.13 | 96.1 ± 4.3 |
| | | (100.00) | (100.00) | (100.00) | |
| Diabetic Control | — | 0.842 ± 0.12 | 0.769 ± 0.07 | 1355.00 ± 25.37 | 590.6 ± 15.1 |
| | | (194.46) | (38.98) | (235.35) | (100) |
| Losartan | 5.5 | 0.808 | 0.827 | 1180.00 | 582.1 ± 00 |
| | | (186.61) | (41.92) | (204.95) | (98.57) |
| Hydroxychloroquine | 22.5 | 0.772 ± 0.06 | 0.827 ± 0.08 | 1098.00 ± 23.12 | 369.9 ± 42.3 |
| | | (178.29) | (41.92) | (190.71) | (62.64)) |
| Losartan + | Losartan (5.5) + | 0.722 ± 0.04 | 0.994 ± 0.15 | 859.17 ± 27.09 | 377.8 ± 12.0 |
| Hydroxychloroquine | Hydroxychloroquine | (166.74) | (50.38) | (149.23) | (63.97) |
| | (22.5) | | | | |

Study 2: Experimental Studies to Evaluate Efficacy of Hydroxychloroquine and its Combination with Losartan in Comparison with Anti-Diabetic Control in Renal Damage

30 Wistar rats of either sex weighing 150-250 g were divided randomly into 5 groups consisting of 6 animals each. Diabetes was induced in rats by single administration of Streptozotocin (STZ)(55 mg/kg/i.p) dissolved in 0.1 M-citrate buffer, pH 4.5. Group 1 (normal control) consisted of normal rats that neither received streptozotocin nor any test drug; Group 2 served as Anti-Diabetic control wherein diabetic rats were treated with antidiabetic drug Glimepiride. Group 3 was diabetic rats treated with Losartan (11 mg/kg); Group 4 was diabetic rats treated with Hydroxychloroquine (45 mg/kg) and Group 5 was diabetic rats treated with combination of Hydroxychloroquine (45 mg/kg) and Losartan (11 mg/kg).

The study drugs were dissolved/suspended in distilled water and administered orally once a day for eight weeks. The diabetes mellitus induced nephropathy was assessed by estimating the level of serum creatinine and total serum protein content after completion of eighth week of study. TBARS and Total Protein were measured as markers of renal oxidative stress in Kidney Homogenate after completion of eighth week of study.

The level of serum creatinine was significantly increased (p<0.01) in antidiabetic control rats as compared to the normal group at the completion of the eighth week (Table 2). In comparison with the antidiabetic control group, the test drugs caused a significant reduction in serum creatinine levels in the following order of efficacy: HCQ-LT combination>HCQ>LT (p<0.01) (Table 2), with the HCQ-LT combination demonstrating a synergistic effect. The total serum protein content was reduced to a significant degree (p<0.01) in antidiabetic control rats as compared to the normal group (Table 2). The test drugs caused a significant increase in total serum protein content in the following order of efficacy: HCQ-LT combination>>HCQ>LT in comparison with the antidiabetic control group (p<0.01) (Table 2), wherein a synergistic effect was evident in HCQ-LT combination.

TABLE 2
Effect of HCQ and its combination with LT on Serum
Creatinine and Total Serum Protein levels in Streptozotocin
(STZ) induced diabetic rats

| | | Serum | Total Serum |
|---|---|---|---|
| | | Creatinine | Protein Levels |
| | | (mg/dl) after 8^{th} | (g/dl) after 8^{th} |
| | Dose | week | week(% |
| Group | (mg/kg) | (% Control) | Control) |
| Normal | — | 1.70 | 6.90 |
| Antidiabetic | Glimepiride | 3.17 | 4.73 |
| Control | (8 mg/kg) | (100) | (100) |
| Losartan | Losartan | 3.00 | 5.45 |
| | (11 mg/kg) + | (94.64) | (113.21) |
| | Glimepiride | | |
| | (8 mg/kg) | | |
| Hydroxy- | Hydroxy- | 2.80 | 5.39 |
| chloroquine | chloroquine (45 | (88.33) | (112.24) |
| | mg/kg) + | | |
| | Glimepiride (8 | | |
| | mg/kg) | | |
| Hydroxy- | Hydroxy- | 2.57 | 6.18 |
| chloroquine + | chloroquine(45 | (81.08) | (123.46) |
| Losartan | mg/kg) + | | |
| | Losartan (11 | | |
| | mg/kg) + | | |
| | Glimepiride (8 | | |
| | mg/kg) | | |

The level of TBARS and Total Protein in Kidney Homogenate were increased to a significant degree (p<0.01) in antidiabetic control rats as compared to the normal group at the completion of the eighth week (Table 3). A synergistic effect was observed with the use of Hydroxychloroquine-Losartan Combination, such that the Total Protein in Kidney Homogenate was reduced to a significant level by test drugs in following order of efficacy: HCQ-LT combination>>LT>HCQ. In comparison with the antidiabetic control group, the test drugs caused a significant decrease in TBARS levels in the following order of efficacy: HCQ>HCQ-LT combination>LT (p<0.01) (Table 3).

TABLE 3
Effect of HCQ and its combination with LT on renal oxidative
stress parameters in Streptozocin (STZ) induced diabetic rats

| | | | Total Protein |
|---|---|---|---|
| | | | in Kidney |
| | Treatment | TBARS(nM/mg) | Homogenate |
| | Dose | Mean(% | (μg/ml) |
| Group | (mg/kg) | Control) | Mean(% Control) |
| Normal | — | 0.27 | 578.17 |
| Diabetic | Glimepiride | 0.82 | 926.67 |
| Nephropathy | (8 mg/kg) | (100) | (100) |
| Control | | | |
| Losartan | Losartan | 0.63 | 909.50 |
| | (11 mg/kg) + | (76.83) | (98.15) |
| | Glimepiride | | |
| | (8 mg/kg) | | |
| Hydroxy- | Hydroxy- | 0.47 | 1003.83 |
| chloroquine | chloroquine(45 | (57.32) | (107.68) |
| | mg/kg) + | | |
| | Glimepiride (8 | | |
| | mg/kg) | | |
| Hydroxy- | Hydroxy- | 0.49 | 643.33 |
| chloroquine + | chloroquine(45 | (59.76) | (69.43) |
| Losartan | mg/kg) + | | |
| | Losartan (11 | | |
| | mg/kg) + | | |
| | Glimepiride (8 | | |
| | mg/kg) | | |

Thus, these results demonstrate for the first time the efficacy of Hydroxychloroquine and the synergistic effect of Hydroxychloroquine-Losartan combination in improvement of biochemical and renal oxidative stress parameters including serum creatinine, blood glucose level, Thiobarbituric acid reactive substances (TBARS), Glutathione (GSH), Total Protein in Kidney Homogenate. A marked improvement in the abovementioned parameters is indicative of recovery from renal damage.

Study 3: Experimental Study to Evaluate Efficacy of Hydroxychloroquine and its Combination with Losartan, Telmisartan, Azilsartan and Ramipril in STZ Induced Diabetic Nephropathy

Male Wistar rats weighing 150-350 g were divided randomly into 29 groups consisting of 6 animals each. Diabetes was induced in rats by single administration of streptozotocin (55 mg/kg/i.p) dissolved in 0.1 M-citrate buffer, pH 4.5. Group 1 (normal control) consisted of normal rats that neither received streptozotocin nor any test drug; Group 2 consisted of normal rats treated with vehicle, Group 3 served as Streptozotocin (STZ) induced diabetic control. Group 4 was diabetic rats treated with Losartan (LT) (5.5 mg/kg); Group 5 was diabetic rats treated with LT (11 mg/kg); Group 6 was diabetic rats treated with Hydroxychloroquine (HCQ) (22.5 mg/kg), Group 7 was diabetic rats treated with HCQ (45 mg/kg); Group 8 was diabetic rats treated with combination of LT (5.5 mg/kg) and HCQ (22.5 mg/kg); Group 9 was diabetic rats treated with combination of LT (5.5 mg/kg) and HCQ (45 mg/kg); Group 10 was diabetic rats treated with LT (11 mg/kg) and HCQ (22.5 mg/kg); Group 11 was diabetic rats treated with LT (11 mg/kg) and HCQ (45 mg/kg); Group 12 was diabetic rats treated with Telmisartan (TL) (5 mg/kg); Group 13 was diabetic rats treated with Telmisartan (TL) (10 mg/kg); Group 14 was diabetic rats treated with the combination of TL (5 mg/kg) and HCQ (22.5 mg/kg); Group 15 was diabetic rats treated with the combination of TL (5 mg/kg) and HCQ (45 mg/kg); Group 16 was diabetic rats treated with the combination of TL (10 mg/kg) and HCQ (22.5 mg/kg); Group 17 was diabetic rats treated with the combination of TL (10 mg/kg) and HCQ (45 mg/kg); Group 18 was diabetic rats treated with Azilsartan (AZ) (2 mg/kg); Group 19 was diabetic rats treated with Azilsartan (AZ) (4 mg/kg); Group 20 was diabetic rats treated with combination of AZ (2 mg/kg) and HCQ (22.5 mg/kg); Group 21 was diabetic rats treated with combination of AZ (2 mg/kg) and HCQ (45 mg/kg); Group 22 was diabetic rats treated with combination of AZ (4 mg/kg) and HCQ (22.5 mg/kg); Group 23 diabetic rats treated with combination of AZ (4 mg/kg) and HCQ (45 mg/kg); Group 24 was diabetic rats treated with Ramipril (RM) (0.2 mg/kg); Group 25 was diabetic rats treated with RM (2.3 mg/kg); Group 26 was diabetic rats treated with combination of RM mg/kg) and HCQ (22.5 mg/kg); Group 27 was diabetic rats treated with combination of RM (0.2 mg/kg) and HCQ (45 mg/kg); Group 28 was diabetic rats treated with combination of RM (2.3 mg/kg) and HCQ (22.5 mg/kg); Group 29 was diabetic rats treated with combination of RM (2.3 mg/kg) and HCQ (45 mg/kg). The study drugs (LT and HCQ) were dissolved in distilled water and other study drugs (AZ, RM and TL) were suspended in 0.5% w/v sodium carboxy methyl cellulose (CMC) and administered orally once a day for eight weeks. Biochemical parameters like serum urea, serum creatinine, serum protein, and renal oxidative stress parameters like TBARS and GSH were assessed after the completion of eighth week of the study.

The levels of TBARS was significantly increased in diabetic rats as compared to normal controls at the completion of eighth week (p<0.01) (Table 4). In comparison with the diabetic control group, the test drugs caused significant reduction in the TBARS levels in the following order of efficacy: HCQ-LT combination>HCQ>LT; HCQ-TL combination>TL>HCQ; HCQ-AZ combination>AZ>HCQ and HCQ-RM combination>HCQ>RM (p<0.05).

The Glutathione (GSH) levels were significantly reduced (p<0.01) in diabetic rats as compared to normal controls at the completion of eighth week (Table 4). In comparison with the diabetic control group, the test drugs caused a significant increase in the Glutathione levels in the following order of efficacy: HCQ-LT combination>HCQ>LT; HCQ-TL combination>HCQ>TL; HCQ-AZ combination>HCQ>AZ and HCQ>HCQ-RM combination>RM (p<0.05) (Table 4).

The level of serum creatinine was significantly increased (p<0.01) in diabetic control rats as compared to the normal group at the completion of the eighth week (Table 4). In comparison with the diabetic control group, the test drugs caused a significant reduction in serum creatinine levels in the following order of efficacy: HCQ-LT combination>HCQ>LT; HCQ-RM combination>HCQ>RM; HCQ-TL combination>HCQ>TL and HCQ>HCQ-AZ combination>AZ (0.05) respectively (Table 4). The total serum protein content was reduced to a significant degree (p<0.01) in diabetic control rats as compared to the normal group (Table 4). The test drugs caused a significant increase in total serum protein content in the following order of efficacy: HCQ-LT combination>HCQ>LT; HCQ-TL combination>TL>HCQ; HCQ-AZ combination>AZ>HCQ; and HCQ>HCQ-RM combination>RM respectively (0.05) (Table 4).

TABLE 4
Effect of hydroxychloroquine and its combination with ARBs (Losartan, Azilsartan,
and Telmisartan) or ACEI (Ramipril) in STZ induced diabetic nephropathy in rats

| | Treatment | Serum creatinine | Serum protein | Serum urea |
|---|---|---|---|---|
| Group | (mg/kg) | (mg/dl) | (g/dl) | (mg/dl) |
| 1 | Naïve | 0.62 ± 0.07 | 7.35 ± 0.36 | 46.11 ± 2.90 |
| 2 | CMC (0.5%) | 0.65 ± 0.07 | 7.45 ± 0.39 | 46.88 ± 1.74 |
| 3 | STZ | 1.41 ± 0.10 | 4.68 ± 0.29 | 107.54 ± 6.36 |
| 4 | LT(5.5) | 1.23 ± 0.18 | 5.23 ± 0.31 | 101.45 ± 2.69 |
| 5 | LT(11) | 0.99 ± 0.05* | 5.75 ± 0.23* | 93.10 ± 4.45* |
| 6 | HCQ(22.5) | 0.87 ± 0.07* | 5.57 ± 0.20 | 91.29 ± 7.29* |
| 7 | HCQ(45) | 0.74 ± 0.12* | 5.95 ± 0.31* | 83.64 ± 6.60* |
| 8 | LT(5.5) + | 0.82 ± 0.05*† | 5.89 ± 0.34* | 84.77 ± 6.40*† |
| | HCQ(22.5) | | | |
| 9 | LT(5.5) + HCQ(45) | 0.74 ± 0.07*†¥ | 6.04 ± 0.62* | 75.72 ± 3.75*†¥ |
| 10 | LT(11) + HCQ(22.5) | 0.83 ± 0.04* | 6.10 ± 0.49* | 78.88 ± 3.35*†¥ |
| 11 | LT(11) + HCQ(45) | 0.66 ± 0.12*†¥ | 6.23 ± 0.37* | 72.58 ± 8.76*†¥ |
| 12 | TL(5) | 1.02 ± 0.13* | 5.94 ± 0.46* | 92.39 ± 4.06* |
| 13 | TL(10) | 0.78 ± 0.07* | 6.53 ± 0.40* | 82.71 ± 6.53* |
| 14 | TL(5) + HCQ(22.5) | 0.96 ± 0.06* | 6.31 ± 0.52* | 82.61 ± 2.80* |
| 15 | TL(5) + HCQ(45) | 0.83 ± 0.07* | 6.80 ± 0.45* | 75.29 ± 3.96*† |
| 16 | TL(10) + HCQ(22.5) | 0.77 ± 0.07* | 6.84 ± 0.76* | 78.88 ± 5.31* |
| 17 | TL(10) + HCQ(45) | 0.62 ± 0.14* | 7.20 ± 0.31* | 70.00 ± 5.07* |
| 18 | AZ(2) | 0.98 ± 0.07* | 5.79 ± 0.37* | 89.83 ± 4.67* |
| 19 | AZ(4) | 0.87 ± 0.04* | 6.76 ± 0.16* | 75.94 ± 4.11* |
| 20 | AZ(2) + HCQ(22.5) | 0.90 ± 0.06* | 5.94 ± 0.49* | 79.24 ± 6.45* |
| 21 | AZ(2) + HCQ(45) | 0.85 ± 0.07* | 6.17 ± 0.56* | 73.87 ± 4.98*† |
| 22 | AZ(4) + HCQ(22.5) | 0.75 ± 0.06* | 7.23 ± 0.46* | 68.54 ± 10.61* |
| 23 | AZ(4) + HCQ(45) | 0.71 ± 0.10* | 7.40 ± 0.32* | 64.41 ± 4.52* |
| 24 | RM(0.2) | 0.90 ± 0.05* | 5.30 ± 0.36 | 98.33 ± 6.15 |
| 25 | RM(2.3) | 0.85 ± 0.06* | 5.67 ± 0.27* | 87.83 ± 5.23* |
| 26 | RM(0.2) + HCQ(22.5) | 0.88 ± 0.07* | 5.69 ± 0.23* | 88.28 ± 4.17* |
| 27 | RM(0.2) + HCQ(45) | 0.82 ± 0.06* | 5.85 ± 0.28* | 88.30 ± 2.69* |
| 28 | RM(2.3) + HCQ(22.5) | 0.84 ± 0.04* | 5.93 ± 0.38* | 78.04 ± 3.46* |
| 29 | RM(2.3) + HCQ(45) | 0.71 ± 0.09* | 6.15 ± 0.44* | 74.86 ± 4.03*†¥ |

| | Creatinine | Urea | | |
|---|---|---|---|---|
| | clearance | clearance | TBARS | GSH |
| Group | (ml/min) | (ml/min) | (nM/mg) | (nM/mg) |
| 1 | 1.06 ± 0.23 | 0.83 ± 0.04 | 0.514 ± 0.11 | 22.24 ± 1.98 |
| 2 | 1.09 ± 0.27 | 0.80 ± 0.06 | 0.571 ± 0.12 | 20.05 ± 2.16 |
| 3 | 0.43 ± 0.04 | 0.27 ± 0.03 | 0.850 ± 0.15 | 7.80 ± 1.19 |
| 4 | 0.59 ± 0.06 | 0.33 ± 0.02 | 0.760 ± 0.05 | 8.66 ± 0.48 |
| 5 | 0.67 ± 0.12* | 0.44 ± 0.03* | 0.670 ± 0.05 | 12.78 ± 1.48* |
| 6 | 0.60 ± 0.06 | 0.36 ± 0.03 | 0.726 ± 0.06 | 8.59 ± 0.76 |
| 7 | 0.67 ± 0.10* | 0.42 ± 0.04* | 0.608 ± 0.06* | 14.29 ± 1.33* |
| 8 | 0.74 ± 0.09* | 0.44 ± 0.06*† | 0.602 ± 0.13* | 10.25 ± 2.03 |
| 9 | 0.77 ± 0.05* | 0.46 ± 0.08*† | 0.588 ± 0.16* | 14.79 ± 1.59* |
| 10 | 0.79 ± 0.05* | 0.45 ± 0.05* | 0.599 ± 0.10* | 15.42 ± 1.60* |
| 11 | 0.80 ± 0.13* | 0.48 ± 0.08* | 0.527 ± 0.07* | 15.84 ± 1.50* |
| 12 | 0.73 ± 0.07* | 0.45 ± 0.05* | 0.675 ± 0.07 | 9.65 ± 1.24 |
| 13 | 0.77 ± 0.04* | 0.47 ± 0.04* | 0.560 ± 0.06* | 13.82 ± 0.34* |
| 14 | 0.75 ± 0.03* | 0.48 ± 0.05* | 0.629 ± 0.08* | 11.01 ± 1.15 |
| 15 | 0.78 ± 0.06* | 0.50 ± 0.05* | 0.576 ± 0.10* | 13.16 ± 1.94* |
| 16 | 0.78 ± 0.04* | 0.53 ± 0.03* | 0.564 ± 0.08* | 14.04 ± 2.84* |
| 17 | 0.81 ± 0.12* | 0.55 ± 0.05* | 0.502 ± 0.06* | 14.47 ± 0.60* |
| 18 | 0.69 ± 0.05* | 0.41 ± 0.07* | 0.624 ± 0.11* | 9.54 ± 1.54 |
| 19 | 0.74 ± 0.04* | 0.44 ± 0.03* | 0.540 ± 0.04* | 13.49 ± 0.90* |
| 20 | 0.77 ± 0.06* | 0.47 ± 0.04* | 0.599 ± 0.18* | 11.35 ± 1.31 |
| 21 | 0.74 ± 0.06* | 0.52 ± 0.05* | 0.564 ± 0.11* | 13.62 ± 2.20*† |
| 22 | 0.88 ± 0.09* | 0.50 ± 0.08* | 0.520 ± 0.02* | 14.42 ± 0.94* |
| 23 | 0.90 ± 0.07* | 0.54 ± 0.05* | 0.512 ± 0.12* | 14.78 ± 2.72* |
| 24 | 0.54 ± 0.05 | 0.41 ± 0.05* | 0.716 ± 0.06 | 8.48 ± 0.59 |
| 25 | 0.61 ± 0.07 | 0.45 ± 0.05* | 0.614 ± 0.06* | 10.57 ± 0.37 |
| 26 | 0.71 ± 0.06* | 0.42 ± 0.04* | 0.609 ± 0.09* | 10.19 ± 1.40 |
| 27 | 0.69 ± 0.07* | 0.43 ± 0.09* | 0.541 ± 0.09* | 10.64 ± 1.65 |
| 28 | 0.69 ± 0.17* | 0.48 ± 0.05* | 0.580 ± 0.07* | 11.42 ± 1.73 |
| 29 | 0.76 ± 0.11* | 0.52 ± 0.03* | 0.535 ± 0.07* | 12.26 ± 2.46* |

*P < 0.05 compared to STZ induced DN group,
†p < 0.05 compared to its respective dose of LT, TL, AZ, RM;
¥p < 0.05 compared to LT (11 mg/kg), TL(10 mg/kg), AZ(4 mg/kg), RM(2.3 mg/kg).

Study 4: Experimental Study to Evaluate Efficacy of Hydroxychloroquine and its Combination with Losartan, Telmisartan, Azilsartan and Ramipril in Streptozotocin (STZ) Induced Diabetic Nephropathy in Rats

Wistar rats of either sex weighing 150-200 g were divided randomly into 28 groups consisting of 6 animals each. Diabetes was induced in rats by single administration of streptozotocin (55 mg/kg/i.p) dissolved in 0.1 M-citrate buffer, pH 4.5. Group 1 (normal control) consisted of normal rats that neither received streptozotocin nor any test drug; Group 2 served as Diabetic control; Group 3 was diabetic rats treated with Hydroxychloroquine (35 mg/kg); Group 4 was diabetic rats treated with Hydroxychloroquine (45 mg/kg) and Group 5 was diabetic rats treated with Hydroxychloroquine (70 mg/kg); Group 6 was diabetic rats treated with Losartan (5.5 mg/kg); Group 7 was diabetic rats treated with Losartan (11 mg/kg); Group 8 was diabetic rats treated with combination of Losartan (5.5 mg/kg) and Hydroxychloroquine (45 mg/kg); Group 9 was diabetic rats treated with combination of Losartan (11 mg/kg) and Hydroxychloroquine (35 mg/kg); Group 10 was diabetic rats treated with Ramipril (0.6 mg/kg); Group 11 was diabetic rats treated with Ramipril (0.3 mg/kg); Group 12 was diabetic rats treated with combination of Ramipril (0.6 mg/kg) and Hydroxychloroquine (35 mg/kg); Group 13 was diabetic rats treated with combination of Ramipril (0.3 mg/kg) and Hydroxychloroquine (45 mg/kg); Group 14 was diabetic rats treated with combination of Ramipril (0.6 mg/kg) and Hydroxychloroquine (45 mg/kg); Group 15 was diabetic rats treated with combination of Ramipril (0.3 mg/kg) and Hydroxychloroquine (35 mg/kg); Group 16 was diabetic rats treated with Telmisartan (9.5 mg/kg); Group 17 was diabetic rats treated with Telmisartan (4.5 mg/kg); Group 18 was diabetic rats treated with combination of Telmisartan (9.5 mg/kg) and Hydroxychloroquine (35 mg/kg); Group 19 was diabetic rats treated with combination of Telmisartan (4.5 mg/kg) and Hydroxychloroquine (45 mg/kg); Group 20 was diabetic rats treated with combination of Telmisartan (9.5 mg/kg) and Hydroxychloroquine (45 mg/kg); Group 21 was diabetic rats treated with combination of Telmisartan (4.5 mg/kg) and Hydroxychloroquine (35 mg/kg); Group 22 was diabetic rats treated with Azilsartan (4.5 mg/kg); Group 23 was diabetic rats treated with Azilsartan (2.5 mg/kg); Group 24 was diabetic rats treated with combination of Azilsartan (4.5 mg/kg) and Hydroxychloroquine (35 mg/kg); Group 25 was diabetic rats treated with combination of Azilsartan (2.5 mg/kg) and Hydroxychloroquine (45 mg/kg); Group 26 was diabetic rats treated with combination of Azilsartan (4.5 mg/kg) and Hydroxychloroquine (45 mg/kg); Group 27 was diabetic rats treated with combination of Azilsartan (2.5 mg/kg) and Hydroxychloroquine (35 mg/kg); Group 28 served as Anti-Diabetic control wherein diabetic rats were treated with antidiabetic drug Glimepiride (GLM) (1 mg/kg).

The study drugs were dissolved/suspended in vehicle and administered orally once a day for eight weeks. The diabetes mellitus induced nephropathy was assessed by estimating the level of serum urea, serum creatinine and total serum protein content after completion of eighth week of study. TBARS, GSH and Total Protein were measured as markers of renal oxidative stress in Kidney Homogenate after completion of eighth week of study. Blood glucose levels were also assessed at weekly interval.

The level of serum creatinine was significantly increased (p<0.01) in antidiabetic control rats as compared to the normal group at the completion of the eighth week (Table 5). In comparison with the antidiabetic control group, the test drugs caused a significant reduction in serum creatinine levels in the following order of efficacy: HCQ-LT combination>HCQ>LT; HCQ-RM combination>HCQ>RM; HCQ-TL combination>HCQ>TL (p<0.05) and HCQ-AZ combination>HCQ>AZ (0.05) respectively (Table 5), with the combination treatments demonstrating a synergistic effect. The total serum protein content was reduced to a significant degree (p<0.01) in antidiabetic control rats as compared to the normal group (Table 5). The test drugs caused a significant increase in total serum protein content in the following order of efficacy: HCQ-LT combination>HCQ>LT; HCQ-RM combination>HCQ>RM; HCQ-TL combination>HCQ>TL (p<0.05) and HCQ-AZ combination>HCQ>AZ (0.05) respectively (Table 5) in comparison with the antidiabetic control group (p<0.01) (Table 5), wherein a synergistic effect was evident in combination treatments.

The level of TBARS and Total Protein in Kidney Homogenate were increased to a significant degree (p<0.01) in antidiabetic control rats as compared to the normal group at the completion of the eighth week (Table 5). In animals treated with combination of HCQ-LT, HCQ-RM and HCQ-AZ the Total Protein in Kidney Homogenate was reduced in following order of efficacy: HCQ-LT combination>LT>HCQ; HCQ-RM>HCQ>RM and HCQ-AZ>HCQ>AZ respectively (p<0.05). The reduction in total protein in kidney homogenate in group treated with combination of HCQ-TL was more than the antidiabetic control group. In comparison with the antidiabetic control group, the Hydroxychloroquine-Telmisartan combination caused a decrease in TBARS levels in the following order of efficacy: HCQ-TL combination>GLM>TL>HCQ (Table 5).

Blood Glucose levels were significantly increased (p<0.01) in diabetic rats as compared to normal control at the completion of eighth week (Table 6). In comparison with the diabetic control group, the test drugs caused a significant decrease in blood glucose levels in the following order of efficacy: HCQ-LT combination>HCQ>LT; HCQ-RM combination>HCQ>RM; HCQ-TL combination>TL>HCQ and HCQ-AZ combination>AZ>HCQ respectively (p<0.05) (Table 6).

TABLE 5
Effect of hydroxychloroquine and its combination with ARBs (Losartan, Azilsartan,
Telmisartan) and ACEI (Ramipril) in STZ induced diabetic nephropathy in rats

| | | Total | | | |
|---|---|---|---|---|---|
| | Treatment | protein | Urea | Creatinine | TC |
| Group | mg/kg | (g/dl) | (mg/dl) | (mg/dl) | (mg/dl) |
| 1 | NORMAL | 7.42 ± 0.48 | 28.67 ± 1.86 | 1.86 ± 0.15 | 105.67 ± 4.27 |
| 2 | DIABETIC | 4.16 ± 0.22 | 49 ± 2.61 | 4 ± 0.2 | 157 ± 7.95 |
| 3 | HCQ (35) | 4.52 ± 0.39 | 44.33 ± 1.75* | 3.11 ± 0.16* | 133.5 ± 3.62*‡ |
| 4 | HCQ (45) | 5.4 ± 0.36* | 40.83 ± 2.14* | 2.99 ± 0.34* | 130.33 ± 2.07*‡ |
| 5 | HCQ (70) | 5.72 ± 0.3*‡ | 38.17 ± 1.47*‡ | 2.68 ± 0.23*‡ | 124.67± 2.94*‡ |
| 6 | LT (5.5) | 4.91 ± 0.2* | 39.83 ± 1.72*‡ | 3.36 ± 0.32* | 143.5 ± 3.39* |
| 7 | LT (11) | 5.32 ± 0.2* | 41 ± 2.53* | 3.26 ± 0.38* | 139.83 ± 3.19* |
| 8 | LT + HCQ | 6.31 ± 0.35*†¥‡ | 30.83 ± 3.31*†¥‡ | 2.63 ± 0.27*†¥‡ | 114.67 ± 3.78*†¥‡ |
| | (5.5 + 45) | | | | |
| 9 | LT + HCQ | 4.89 ± 0.26* | 33.67 ± 2.5*†‡ | 3.01 ± 0.22* | 123.67 ± 4.13*†‡ |
| | (11 + 35) | | | | |
| 10 | RM (0.6) | 4.77 ± 0.21 | 41 ± 1.79* | 3.68 ± 0.24* | 142.17 ± 3.31* |
| 11 | RM (0.3) | 4.76 ± 0.28 | 41.33 ± 1.97* | 3.37 ± 0.32* | 138.33 ± 2.94* |
| 12 | RM + HCQ | 5.38 ± 0.25* | 37.83 ± 2.32*†‡ | 3.16 ± 0.31*† | 137.17 ± 2.86*†‡ |
| | (0.6 + 35) | | | | |
| 13 | RM + HCQ | 5.21 ± 0.21* | 37.33 ± 3.27*†¥‡ | 3.22 ± 0.23*¥ | 131.67 ± 3.88*†¥‡ |
| | (0.3 + 45) | | | | |
| 14 | RM + HCQ | 6.15 ± 0.4*†‡ | 32.5 ± 2.07*†‡ | 3.03 ± 0.25*† | 123.33 ± 2.5*†‡ |
| | (0.6 + 45) | | | | |
| 15 | RM + HCQ | 4.98 ± 0.32* | 35 ± 1.9*†¥‡ | 3.02 ± 0.15*†¥ | 138.33 ± 2.07* |
| | (0.3 + 35) | | | | |
| 16 | TL (9.5) | 4.61 ± 0.18 | 41.5 ± 2.35* | 3.91 ± 0.15‡ | 141.5 ± 2.88* |
| 17 | TL (4.5) | 4.93 ± 0.23* | 42.67 ± 2.5* | 3.68 ± 0.31*‡ | 138.67 ± 3.93* |
| 18 | TL + HCQ | 5.71 ± 0.37*†‡ | 37.33 ± 1.86*†¥‡ | 3.22 ± 0.24*† | 139 ± 1.67* |
| | (9.5 + 35) | | | | |
| 19 | TL + HCQ | 5.32 ± 0.24*¥ | 36 ± 1.55*†‡ | 3.3 ± 0.24*†¥ | 133.33 ± 2.42*†¥‡ |
| | (4.5 + 45) | | | | |
| 20 | TL + HCQ | 5.2 ± 0.2* | 34.17 ± 1.72*†‡ | 2.73 ± 0.22*†‡ | 131.67 ± 3.27*†‡ |
| | (9.5 + 45) | | | | |
| 21 | TL + HCQ | 4.89 ± 0.22* | 38.17 ± 2.64*†¥‡ | 3.18 ± 0.19*†¥ | 137.33 ± 1.75*¥‡ |
| | (4.5 + 35) | | | | |
| 22 | AZ (4.5) | 4.99 ± 0.25 | 41 ± 3.58* | 3.44 ± 0.28* | 141.17 ± 2.32* |
| 23 | AZ (2.5) | 4.76 ± 0.15* | 40.83 ± 2.64* | 3.61 ± 0.16*‡ | 142 ± 2.1* |
| 24 | AZ + HCQ | 5.96 ± 0.26*†‡ | 35.83 ± 2.79*†‡ | 2.73 ± 0.19*†‡ | 138.33 ± 1.75* |
| | (4.5 + 35) | | | | |
| 25 | AZ + HCQ | 5.27 ± 0.25* | 38 ± 3.41*†‡ | 2.77 ± 0.39*†‡ | 138.17 ± 3.6* |
| | (2.5 + 45) | | | | |
| 26 | AZ + HCQ | 6.02 ± 0.46*†‡ | 26.8 ± 1.87*†‡ | 2.61 ± 0.21*†‡ | 139.33 ± 4.59* |
| | (4.5 + 45) | | | | |
| 27 | AZ + HCQ | 5.2 ± 0.27* | 41.17 ± 2.32* | 3.08 ± 0.18*† | 139 ± 4.47* |
| | (2.5 + 35) | | | | |
| 28 | GLM (1) | 4.8 ± 0.18* | 43 ± 2.1* | 3.21 ± 0.23* | 142 ± 2.68* |

| | | | | | Total protein |
|---|---|---|---|---|---|
| | HDL | TG | TBARS | GSH | kidney |
| Group | (mg/dl) | (mg/dl) | (nM/mg) | (nM/mg) | (μg/ml) |
| 1 | 36.5 ± 2.88 | 76.5 ± 4.89 | 0.26 ± 0.08 | 2.12 ± 0.87 | 577.33 ± 20.33 |
| 2 | 25.67 ± 2.25 | 107 ± 3.9 | 0.84 ± 0.07 | 0.6 ± 0.16 | 926.17 ± 45.15 |
| 3 | 29.5 ± 2.17* | 94.83 ± 2.48* | 0.67 ± 0.11* | 0.71 ± 0.16 | 865.33 ± 27.9* |
| 4 | 29 ± 2.19* | 96.83 ± 2.93* | 0.65 ± 0.13* | 0.7 ± 0.17 | 831.67 ± 24.15* |
| 5 | 30.67 ± 1.86* | 90.17 ± 3.49*‡ | 0.63 ± 0.12* | 0.67 ± 0.19 | 752.33 ± 18.8*‡ |
| 6 | 27.5 ± 1.64 | 100 ± 5.4* | 0.74 ± 0.1 | 0.56 ± 0.23 | 893.33 ± 29.92* |
| 7 | 28.5 ± 2.88* | 98.83 ± 3.66* | 0.63 ± 0.17* | 0.66 ± 0.23 | 829.67 ± 25.45* |
| 8 | 32.5 ± 2.07*†¥‡ | 85.33 ± 4.13*†¥‡ | 0.66 ± 0.12* | 1.37 ± 0.59*¥† | 646.67 ± 20.87*†¥‡ |
| 9 | 30.5 ± 1.87* | 93.17 ± 3.49*† | 0.53 ± 0.14* | 1.36 ± 0.49*† | 724.33 ± 19.06*†‡ |
| 10 | 27.33 ± 1.63 | 98.5 ± 4.93* | 0.62 ± 0.15* | 1.16 ± 0.59* | 810 ± 17.12* |
| 11 | 27.17 ± 1.47 | 100.17 ± 5* | 0.67 ± 0.1*‡ | 1.16 ± 0.62* | 835.33 ± 22.27* |
| 12 | 30.83 ± 2.14*† | 99 ± 4.94* | 0.61 ± 0.15* | 1.44 ± 0.61* | 676.83 ± 20.13*†‡ |
| 13 | 30.33 ± 1.97*†¥ | 93.83 ± 3.06*†¥ | 0.66 ± 0.16*‡ | 1.49 ± 0.77* | 781.17 ± 14.59*†‡ |
| 14 | 32 ± 2.37*† | 96.83 ± 2.64* | 0.56 ± 0.14* | 1.91 ± 0.91*† | 691.67 ± 23.43*†‡ |
| 15 | 27.17 ± 2.48 | 92 ± 3.46*†¥ | 0.57 ± 0.13* | 0.86 ± 0.06 | 831.33 ± 26.82* |
| 16 | 26.83 ± 1.47‡ | 97.67 ± 6.09* | 0.54 ± 0.14* | 0.96 ± 0.11 | 864.5 ± 25.65 |
| 17 | 26 ± 1.67‡ | 100.33 ± 5.57* | 0.65 ± 0.15* | 0.86 ± 0.06 | 879.17 ± 29.41 |
| 18 | 26.5 ± 1.64‡ | 100.17 ± 4.71* | 0.48 ± 0.14* | 1.16 ± 0.27* | 754 ± 20.48* |
| 19 | 28.83 ± 2.14*† | 93 ± 3.46*†¥ | 0.57 ± 0.1* | 1.6 ± 0.39*†¥ | 808.33 ± 25.13* |
| 20 | 29.67 ± 1.75*† | 94.67 ± 4.27* | 0.44 ± 0.08* | 1.81 ± 0.63*† | 805.5 ± 12.31* |
| 21 | 29 ± 1.79*†¥ | 99.33 ± 3.78* | 0.59 ± 0.1* | 1.94 ± 0.65*†¥ | 830 ± 21.97* |
| 22 | 26.67 ± 1.51‡ | 103 ± 3.9 | 0.58 ± 0.14* | 1.26 ± 0.44* | 837.5 ± 20.15* |
| 23 | 27.17 ± 1.17 | 97 ± 5.66* | 0.6 ± 0.11* | 0.86 ± 0.21 | 874.5 ± 21.80 |
| 24 | 30.17 ± 1.47*† | 92 ± 2.28*† | 0.57 ± 0.13* | 1.13 ± 0.44 | 652.33 ± 17.42*†‡ |
| 25 | 30.67 ± 1.63*† | 97.83 ± 2.86* | 0.55 ± 0.12* | 1.5 ± 0.52*† | 670 ± 25.27*†‡ |
| 26 | 29 ± 1.55* | 98.5 ± 5.17* | 0.49 ± 0.09* | 2.23 ± 0.59*† | 651.67 ± 25.07*†‡ |
| 27 | 27 ± 1.41 | 96.83 ± 4.07* | 0.57 ± 0.08* | 1.72 ± 0.47*† | 873.67 ± 19.42* |
| 28 | 29 ± 1.41* | 96.5 ± 2.35* | 0.5 ± 0.13* | 2.36 ± 0.64* | 856.83 ± 18.18* |

*P < 0.05 compared to diabetic group,
†p < 0.05 compared to its respective dose of LT, TL, AZ, RM;
¥p < 0.05 compared to high dose LT (11 mg/kg), TL(9.5 mg/kg), AZ(4.5 mg/kg), RM(0.6 mg/kg)
‡p < 0.05 compared to (GLM).

TABLE 6
Effect of hydroxychloroquine and its combination with ARBs (Losartan, Azilsartan and Telmisartan)
and ACEI (Ramipril) on blood glucose levels in STZ induced diabetic nephropathy in rats

| | | Glucose | Glucose | Glucose | Glucose | Glucose |
|---|---|---|---|---|---|---|
| | Treatment | Day1 | Day14 | Day28 | Day42 | Day56 |
| Group | (mg/kg) | (mg/dl) | (mg/dl) | (mg/dl) | (mg/dl) | (mg/dl) |
| 1 | NORMAL | 88 ± 8.32 | 88.33 ± 4.84 | 87.83 ± 5.67 | 84.83 ± 4.58 | 86.33 ± 4.97 |
| 2 | DIABETIC | 262.67 ± 10.58 | 271.5 ± 9.44 | 270.5 ± 8.76 | 285.17 ± 7.19 | 302.67 ± 21.57 |
| 3 | HCQ (35) | 268.83 ± 17.77 | 203.33 ± 28.11* | 176.83 ± 29.1* | 165 ± 13.61* | 151.5 ± 7.42* |
| 4 | HCQ (45) | 277.5 ± 17.74 | 229.17 ± 10.38* | 184.17 ± 14.5* | 154.17 ± 9.13* | 140 ± 7.56* |
| 5 | HCQ (70) | 274.5 ± 16.01 | 209.67 ± 21.69* | 169.5 ± 10.48* | 148.67 ± 8.5* | 130.83 ± 7.22*‡ |
| 6 | LT (5.5) | 262.5 ± 25.35 | 211.17 ± 21.48* | 179.33 ± 7.84* | 161.17 ± 4.71* | 146.33 ± 6.65* |
| 7 | LT (11) | 268.67 ± 15.63 | 215.67 ± 18.13* | 178 ± 7.92* | 152.17 ± 7.28* | 139 ± 10.92* |
| 8 | LT + HCQ | 265.83 ± 16.42 | 213.33 ± 20.6* | 172.33 ± 16.12* | 148.5 ± 9.79*† | 121.5 ± 10.25*†‡ |
| | (5.5 + 45) | | | | | |
| 9 | LT + HCQ | 272 ± 15.74 | 219 ± 16.2* | 181 ± 8.17* | 155.5 ± 6.5* | 136.33 ± 6.8* |
| | (11 + 35) | | | | | |
| 10 | RM (0.6) | 260 ± 17.19 | 203 ± 14.07* | 174.33 ± 8.91* | 157.67 ± 9.18* | 141.67 ± 5.47* |
| 11 | RM (0.3) | 281.5 ± 18.2 | 216.17 ± 21.48* | 174.17 ± 9.06* | 158.5 ± 9.07* | 145.33 ± 6.38* |
| 12 | RM + HCQ | 264.67 ± 17.45 | 209 ± 19.87* | 177.33 ± 8.96* | 149.33 ± 5.57* | 134.67 ± 6.15* |
| | (0.6 + 35) | | | | | |
| 13 | RM + HCQ | 265 ± 14.04 | 207.5 ± 15.76* | 176.5 ± 7.23* | 152 ± 6.07* | 132.17 ± 7.36* |
| | (0.3 + 45) | | | | | |
| 14 | RM + HCQ | 268.33 ± 14.17 | 209.5 ± 12.08* | 186 ± 8.29* | 152.5 ± 8.38* | 128.83 ± 6.77*† |
| | (0.6 + 45) | | | | | |
| 15 | RM + HCQ | 289.33 ± 10.05 | 229 ± 10.3* | 190 ± 7.46* | 158.5 ± 6.09*¥ | 138.5 ± 6.92*† |
| | (0.3 + 35) | | | | | |
| 16 | TL (9.5) | 275.33 ± 15.45 | 221.5 ± 16.02 | 184.33 ± 8.31 | 159.83 ± 7.03 | 141 ± 7.07 |
| 17 | TL (4.5) | 281.83 ± 12.46 | 221.83 ± 12.37 | 181.83 ± 5.04 | 162 ± 9.21 | 139 ± 7.85 |
| 18 | TL + HCQ | 276.5 ± 12.63 | 216.83 ± 13.3 | 186.67 ± 6.95 | 158.5 ± 6.53 | 140.5 ± 6.09 |
| | (9.5 + 35) | | | | | |
| 19 | TL + HCQ | 270.83 ± 13.88 | 215 ± 11.01 | 183 ± 5.22 | 162 ± 5.8 | 136.17 ± 6.21 |
| | (4.5 + 45) | | | | | |
| 20 | TL + HCQ | 278.5 ± 12.77 | 222.33 ± 16.19 | 178.5 ± 7.06 | 153 ± 6.84 | 130.5 ± 6.63 |
| | (9.5 + 45) | | | | | |
| 21 | TL + HCQ | 272.67 ± 18.51 | 219.33 ± 15 | 180.67 ± 8.45 | 157.5 ± 6.57 | 137.33 ± 5.57 |
| | (4.5 + 35) | | | | | |
| 22 | AZ (4.5) | 278.5 ± 14.22 | 224.17 ± 17.93* | 179 ± 7.13* | 158.67 ± 6.77* | 139.83 ± 4.79* |
| 23 | AZ (2.5) | 267.17 ± 12.21 | 215.5 ± 14.01* | 180 ± 8.00* | 160.67 ± 6.12* | 141 ± 6.924* |
| 24 | AZ + HCQ | 272.5 ± 10.71 | 213.5 ± 14.95* | 179.17 ± 5.49* | 155.83 ± 4.83* | 138.5 ± 7.77* |
| | (4.5 + 35) | | | | | |
| 25 | AZ + HCQ | 268.17 ± 14.62 | 213.17 ± 15.84* | 185.67 ± 6.41*¥ | 158.5 ± 5.79* | 139.5 ± 8.69* |
| | (2.5 + 45) | | | | | |
| 26 | AZ + HCQ | 288.67 ± 16.9 | 224.17 ± 16.69* | 183.83 ± 7.94* | 158 ± 6.51* | 138.83 ± 7.65*‡ |
| | (4.5 + 45) | | | | | |
| 27 | AZ + HCQ | 267 ± 12.84 | 209.5 ± 12.01* | 182.67 ± 5.43* | 163.33 ± 6.41* | 143.33 ± 8.59* |
| | (2.5 + 35) | | | | | |
| 28 | GLM (1) | 276.83 ± 19.14 | 216.67 ± 18.44* | 179.17 ± 7.36* | 159 ± 6.07* | 149.17 ± 7.83* |

*P < 0.05 compared to diabetic group,
†p < 0.05 compared to its respective dose of LT, TL, AZ, RM;
¥p < 0.05 compared to high dose LT (11 mg/kg), TL(10 mg/kg), AZ(4 mg/kg), RM(0.6 mg/kg);
‡p < 0.05 compared to (GLM).

Thus, these results demonstrate for the first time the efficacy of Hydroxychloroquine and the synergistic effect of combination of Hydroxychloroquine with ARBs (Losartan, Azilsartan, Telmisartan) and ACEI (Ramipril) in improvement of blood glucose levels, biochemical including serum protein, serum creatinine and renal oxidative stress parameters including Thiobarbituric Acid Reactive Substances (TBARS), Glutathione (GSH), Total Protein in Kidney Homogenate. A marked improvement in the above mentioned parameters is indicative of recovery from renal damage.

### Example 1

i. Each tablet/capsule contains:

| | |
|---|---|
| Hydroxychloroquine | 100 mg |
| Losartan | 25 mg/50 mg |
| Excipients q.s. | to make 400 mg tablet/capsule |

ii. Each tablet/capsule contains:

| | |
|---|---|
| Hydroxychloroquine | 200 mg |
| Losartan | 25 mg/50 mg |
| Excipients q.s. | to make 500 mg tablet/capsule |

iii. Each tablet/capsule contains:

| | |
|---|---|
| Hydroxychloroquine | 300 mg |
| Losartan | 50 mg/100 mg |
| Excipients q.s. | to make 700 mg tablet/capsule |

iv. Each tablet/capsule contains:

| | |
|---|---|
| Hydroxychloroquine | 400 mg |
| Losartan | 25 mg/50 mg |
| Excipients q.s. | to make 800 mg tablet/capsule |

### Example 2

i. Each tablet/capsule contains:

| | |
|---|---|
| Hydroxychloroquine | 100 mg |
| Azilsartan | 20 mg/40 mg |
| Excipients q.s. | to make 400 mg tablet/capsule |

ii. Each tablet/capsule contains:

| | |
|---|---|
| Hydroxychloroquine | 200 mg |
| Azilsartan | 20 mg/40 mg |
| Excipients q.s. | to make 500 mg tablet/capsule |

iii. Each tablet/capsule contains:

| | |
|---|---|
| Hydroxychloroquine | 300 mg |
| Azilsartan | 40 mg |
| Excipients q.s. | to make 600 mg tablet/capsule |

iv. Each tablet/capsule contains:

| | |
|---|---|
| Hydroxychloroquine | 400 mg |
| Azilsartan | 40 mg |
| Excipients q.s. | to make 800 mg tablet/capsule |

### Example 3

i. Each tablet/capsule contains:

| | |
|---|---|
| Hydroxychloroquine | 100 mg |
| Ramipril | 2.5 mg/5 mg |
| Excipients q.s. | to make 400 mg tablet/capsule |

ii. Each tablet/capsule contains:

| | |
|---|---|
| Hydroxychloroquine | 200 mg |
| Ramipril | 2.5 mg/5 mg |
| Excipients q.s. | to make 500 mg tablet/capsule |

iii. Each tablet/capsule contains:

| | |
|---|---|
| Hydroxychloroquine | 300 mg |
| Ramipril | 5 mg/10 mg |
| Excipients q.s. | to make 600 mg tablet/capsule |

iv. Each tablet/capsule contains:

| | |
|---|---|
| Hydroxychloroquine | 400 mg |
| Ramipril | 5 mg/10 mg |
| Excipients q.s. | to make 800 mg tablet/capsule |

### Example 4

i. Each tablet/capsule contains:

| | |
|---|---|
| Hydroxychloroquine | 100 mg |
| Lisinopril | 5 mg |
| Excipients q.s. | to make 400 mg tablet/capsule |

ii. Each tablet/capsule contains:

| | |
|---|---|
| Hydroxychloroquine | 200 mg |
| Lisinopril | 10 mg |
| Excipients q.s. | to make 500 mg tablet/capsule |

iii. Each tablet/capsule contains:

| | |
|---|---|
| Hydroxychloroquine | 300 mg |
| Lisinopril | 20 mg |
| Excipients q.s. | to make 600 mg tablet/capsule |

iv. Each tablet/capsule contains:

| | |
|---|---|
| Hydroxychloroquine | 400 mg |
| Lisinopril | 20 mg |
| Excipients q.s. | to make 800 mg tablet/capsule |

### Example 5

i. Each tablet/capsule contains:

| | |
|---|---|
| Hydroxychloroquine | 100 mg |
| Benazepril | 5 mg |
| Excipients q.s. | to make 400 mg tablet/capsule |

ii. Each tablet/capsule contains:

| | |
|---|---|
| Hydroxychloroquine | 200 mg |
| Benazepril | 10 mg |
| Excipients q.s. | to make 500 mg tablet/capsule |

iii. Each tablet/capsule contains:

| | |
|---|---|
| Hydroxychloroquine | 300 mg |
| Benazepril | 20 mg |
| Excipients q.s. | to make 600 mg tablet/capsule |

iv. Each tablet/capsule contains:

| | |
|---|---|
| Hydroxychloroquine | 400 mg |
| Benazepril | 20 mg |
| Excipients q.s. | to make 800 mg tablet/capsule |

It will be evident to those skilled in the art that the invention is not limited to the details of the foregoing illustrative examples and that the present invention may be embodied in other specific forms without departing from the essential attributes thereof, and it is therefore desired that the present embodiments and examples be considered in all respects as illustrative and not restrictive, reference being made to the appended claims, rather than to the foregoing description, and all changes which come within the meaning and range of equivalency of the claims are therefore intended to be embraced therein.

1. A method of treatment or prophylaxis of kidney disease in a human in need thereof, said method comprising administering to the human Hydroxychloroquine, an enantiomer of Hydroxychloroquine, or a pharmaceutically acceptable salt of Hydroxychloroquine or an enantiomer of Hydroxychloroquine, either alone or concomitantly or sequentially with at least one drug selected from the group of Angiotensin receptor blockers and Angiotensin converting enzyme inhibitors.

2. The method of claim 1, wherein the kidney disease is of non-inflammatory etiology.

3. The method of claim 1, wherein the kidney disease comprises kidney disease caused by Type 1 and Type 2 Diabetes Mellitus, Systemic infections, Drug-Induced Nephropathies, Generalized Tubulopathies, Fanconi Syndrome, Acute Tubular Necrosis, Tubular Atrophy, Hypertension, Hypertensive Nephropathy or Nephrosclerosis, Hyperlipidemic Nephropathy or Nephrosclerosis, Renal Artery Disease, Microangiopathy and Idiopathic Kidney Diseases.

4. The method of claim 1, wherein the kidney disease is one or more of an abnormal albumin:creatinine ratio, albuminuria, microalbuminuria, macroalbuminuria, abnormal glomerular filtration rate, higher serum creatinine levels, higher total protein in kidney homogenate, increased blood urea nitrogen levels, hypoproteinemia, abnormal urinary sediment, abnormal results on kidney imaging studies, end stage renal failure.

5. The method of claim 1, wherein the kidney disease is caused by Diabetes Mellitus.

6. The method of claim 1, wherein the kidney disease is caused by Hypertension or Hyperlipidemia.

7. The method of claim 1, wherein the kidney disease is a hypertensive nephropathy.

8. The method of claim 7, wherein the hypertensive nephropathy is caused by arterial hypertension.

9. The method of claim 7, wherein the hypertensive nephropathy is interstitial fibrosis, glomerular alterations or periglomerular fibrosis.

10. The method of claim 1, wherein the kidney disease is one of hyperlipidemic nephropathy and dyslipidemic nephropathy.

11. The method of claim 10, wherein either the hyperlipidemic nephropathy and dyslipidemic nephropathy is caused by persistent filtration of lipids or lipoproteins.

12. The method of claim 10, wherein either the hyperlipidemic nephropathy and dyslipidemic nephropathy is associated with glomerulosclerosis or nephrosis.

13. The method of claim 1, wherein the prophylaxis comprises primary and secondary prophylaxis.

14. The method of claim 1, wherein the Angiotensin Receptor Blocker is one of Valsartan, Telmisartan, Losartan, Irbesartan, Azilsartan, and Olmesartan.

15. The method of claim 1, wherein the Hydroxychloroquine, the enantiomer of Hydroxychloroquine, or the pharmaceutically acceptable salt of Hydroxychloroquine or the enantiomer of Hydroxychloroquine is administered either concomitantly or sequentially with Losartan.

16. The method of claim 1, wherein the Hydroxychloroquine, the enantiomer of Hydroxychloroquine, or the pharmaceutically acceptable salt of Hydroxychloroquine or the enantiomer of Hydroxychloroquine is administered either concomitantly or sequentially with Azilsartan.

17. The method of claim 1, wherein the Angiotensin Converting Enzyme Inhibitor is one of Ramipril, Lisinopril, Perindopril, Captopril, Enalapril, Quinapril, Benazepril, Imidapril, Zofenopril, and Trandolapril.

18. The method of claim 1, wherein the Hydroxychloroquine, the enantiomer of Hydroxychloroquine, or the pharmaceutically acceptable salt of Hydroxychloroquine or the enantiomer of Hydroxychloroquine is administered either concomitantly or sequentially with Ramipril.

19. The method of claim 1, wherein the Hydroxychloroquine, the enantiomer of Hydroxychloroquine, or the pharmaceutically acceptable salt of Hydroxychloroquine or the enantiomer of Hydroxychloroquine is administered either concomitantly or sequentially with Lisinopril.

20. The method of claim 1, wherein the Hydroxychloroquine, the enantiomer of Hydroxychloroquine, or the pharmaceutically acceptable salt of Hydroxychloroquine or the enantiomer of Hydroxychloroquine is administered either concomitantly or sequentially with Benazepril.

21. The method of claim 1, wherein the administration of Hydroxychloroquine, the enantiomer of Hydroxychloroquine, or the pharmaceutically acceptable salt of Hydroxychloroquine or the enantiomer of Hydroxychloroquine ranges from 50-800 mg per day.

22. The method of claim 15, wherein the administration of Losartan ranges from 20-100 mg per day.

23. The method of claim 16, wherein the administration of Azilsartan ranges from 10-100 mg per day.

24. The method of claim 18, wherein the administration of Ramipril ranges from 1-100 mg per day.

25. The method of claim 19, wherein the administration of Lisinopril ranges from 1-100 mg per day.

26. The method of claim 20, wherein the administration of Benazepril ranges from 1-100 mg per day.

27. The method of claim 1, wherein the Hydroxychloroquine, the enantiomer of Hydroxychloroquine, or the pharmaceutically acceptable salt of Hydroxychloroquine or the enantiomer of Hydroxychloroquine is administered by one of an oral, intravenous, intramuscular, subcutaneous, intranasal, intralesional, rectal and topical route of administration.

28. A pharmaceutical combination comprising Hydroxychloroquine, an enantiomer of Hydroxychloroquine, a pharmaceutically acceptable salt of Hydroxychloroquine, or a pharmaceutically acceptable salt of an enantiomer of Hydroxychloroquine, for the treatment or prophylaxis of kidney disease in a human in need thereof.

29. The pharmaceutical combination according to claim 28, wherein the kidney disease is noninflammatory etiology.

30. The pharmaceutical combination according to claim 28, wherein the pharmaceutical combination is administered at a dosage of 50-800 mg/day.

31. The pharmaceutical combination according to claim 28, wherein the combination further comprises at least one drug selected from the group consisting of Angiotensin receptor blockers and Angiotensin converting enzyme inhibitors.

32. The pharmaceutical combination according to claim 31, wherein the Angiotensin Receptor Blockers is one of Valsartan, Telmisartan, Losartan, Irbesartan, Azilsartan, and Olmesartan.

33. The pharmaceutical combination according to claim 31, wherein the Angiotensin Converting Enzyme Inhibitors is one of Ramipril, Lisinopril, Perindopril, Captopril, Enalapril, Quinapril, Benazepril, Imidapril, Zofenopril, and Trandolapril.

34. The pharmaceutical combination according to claim 31, in the form of a fixed dosage combination or a kit, wherein the kit comprises a fixed dosage combination of the Hydroxychloroquine, an enantiomer of Hydroxychloroquine, a pharmaceutically acceptable salt of the Hydroxychloroquine, or a pharmaceutically acceptable salt of the enantiomer of Hydroxoxychloroquine, and an Angiotensin Receptor Blocker.

35. The pharmaceutical combination according to claim 31, in the form of a fixed dosage combination or a kit comprising a fixed dosage combination.

36. A pharmaceutical combination comprising Hydroxychloroquine, an enantiomer of Hydroxychloroquine, a pharmaceutically acceptable salt of Hydroxychloroquine, or a pharmaceutically acceptable salt of an enantiomer of Hydroxychloroquine, and at least one drug selected from the group consisting of Angiotensin receptor blockers and Angiotensin converting enzyme inhibitors for the treatment or prophylaxis of kidney disease in a human in need thereof.

37. The pharmaceutical combination according to claim 36, wherein the kidney disease is of non-inflammatory etiology.

38. A pharmaceutical combination comprising Hydroxychloroquine, an enantiomer of Hydroxychloroquine, a pharmaceutically acceptable salt of Hydroxychloroquine, or a pharmaceutically acceptable salt of an enantiomer of Hydroxychloroquine, either alone or concomitantly or sequentially in combination with at least one drug selected from the group of Angiotensin receptor blocker and Angiotensin converting enzyme inhibitor for use in the treatment or prophylaxis of kidney disease in a human in need thereof.

39. The pharmaceutical combination according to claim 38, wherein the kidney disease is of non-inflammatory etiology.
